(19) United States
(12) Patent Application Publication
(10) Pub. No.: US 2016/0354503 A1
(43) Pub. Date: Dec. 8, 2016

(54) ULTRAVIOLET DISINFECTION OF MEDICAL DEVICE ACCESS SITES

(71) Applicants: OREGON HEALTH & SCIENCE UNIVERSITY, Portland, OR (US); BAKER GROUP, LLP, Portland, OR (US)

(72) Inventors: Michael Hutchens, Portland, OR (US); Michael J. Baker, Portland, OR (US); Christian Terry Proch McMechan, Victoria (CA); Mark Sasha Drlik, Victoria (CA); Paul Charlebois, Victoria (CA)

(21) Appl. No.: 15/105,841

(22) PCT Filed: Dec. 17, 2014
(86) PCT No.: PCT/US2014/070980

§ 371 Date: Jun. 17, 2016

Related U.S. Application Data
(60) Provisional application No. 61/916,876, filed on Dec. 17, 2013.

(51) Int. Cl. A61L 2/10; A61M 39/16

(52) U.S. Cl. CPC A61L 2/10; A61M 39/16; A61M 2039/167

(57) ABSTRACT

Devices and methods for automatically maintaining disinfection of access sites of medical devices using ultraviolet light are disclosed. In one example approach, a device comprises a controller and an ultraviolet light source incorporated into a chamber of an enclosure which is closeable over access ports. In response to an adjustment of the enclosure from an open position to a closed position, the controller illuminates the ultraviolet light source for a predetermined first duration while the enclosure is maintained in the closed position. Following illumination of the ultraviolet light source for the first duration, the controller illuminates the ultraviolet light source for a predetermined second duration at predetermined time intervals while the enclosure is maintained in the closed position.

### CROSS REFERENCE TO RELATED APPLICATIONS

The present application claims priority to U.S. Provisional Patent Application No. 61/916,876, filed Dec. 17, 2013, entitled “ULTRAVIOLET DISINFECTION OF MEDICAL DEVICE ACCESS SITES,” the entire disclosure of which is hereby incorporated by reference in its entirety.

### FIELD

The present disclosure relates to the field of sterilization or disinfection systems and methods.

### BACKGROUND

Medical devices may include access sites for the administration of fluids, nutrients, medications, and blood products to patients. For example, central venous access using a catheter or central line is a common medical practice used in hospitals to deliver medication to a patient and perform various treatments and/or laboratory tests. As another example, dialysis catheters may be used for exchanging blood to and from a hemodialysis machine from the patient. Many hospitalized patients, particularly intensive care and perioperative patients, are provided with indwelling catheters for medication delivery and/or monitoring. In central venous access, for example, a central venous catheter is placed in a large vein, e.g., in the neck, shoulder, or groin, to permit repeated direct access via an external access site to the bloodstream of the patient. Such catheters bypass natural barriers, offer direct access to the circulation, and often remain in the patient's body for a week or more.

Such access sites may become contaminated during routine use when the access site is exposed to the environment, interfaces with delivery mechanisms, is manipulated by healthcare personnel, etc. Further, entry points of medical devices in a patient, e.g., an entry point of a catheter in the skin of a patient, may also become contaminated. Contamination of access sites and entry points may give rise to harmful infections which arise from microorganisms, such as bacteria, entering the body of a patient via the access sites. For example, if a port in a central venous catheter or a dialysis catheter inserted into a patient becomes contaminated, then an infection, such as central venous catheter-associated bloodstream infection (CLABSI), may occur when fluids are introduced into the bloodstream of the patient via the contaminated port. Catheter ports may be repeatedly used, e.g., accessed 10-30 times daily, and may remain in place for relatively long periods of time thereby increasing risk of infection from contaminated injection ports. Such infections may lead to increased morbidity and mortality, and increased health care costs. For example, post-operative infections may lead to longer hospital stays which are costly for both patients and health care providers. Because of this, it is desirable for hospital staff and physicians to keep access sites disinfected or sufficiently sterilized.

Approaches for sterilizing or disinfecting access sites in order to attempt to reduce infections include chemical disinfection, antibiotic port flushes, and antibiotic coatings. However, antibiotic flushes and coatings may increase the incidence of resistant infection and expose patients to the risk of allergic reactions, which are potentially lethal. In some approaches, after catheter placement, isopropyl alcohol swabs may be used to sanitize injection ports of the catheter prior to each use in a process called “scrubbing the hub.” However, such an approach may be inadequate or ineffective due to the effort and time it takes to sufficiently scrub the hub in order to achieve adequate sanitization. Since practitioners may access catheters or other access sites many times, such an intervention imposes hardship, reduces practitioner availability for other tasks, and is likely underperformed in emergencies.

The issues identified above are not necessarily admitted to be well known and are recognized by the inventors of the present application.

### SUMMARY

The present disclosure is directed to devices and methods for maintaining disinfection of access sites of medical devices, such as ports of catheters or other medical tubing, using an ultraviolet (UV) light source which is selectively illuminated during various conditions in order keep the access sites sterile and ready for use. UV light sterilizes materials by using a wavelength of light that breaks the molecular bonds in microbe DNA which either destroys them, rendering them harmless, or prohibits their growth and reproduction thus taking away their ability to cause infection.

In one example approach, a device may comprise a power supply, e.g., one or more batteries, and a UV light source, e.g., one or more UV light-emitting diodes, incorporated into a chamber of an enclosure which is closeable over one or more access ports to cover or enclose the access ports within the chamber. For example, such an enclosure may include a sealing member, e.g., a door, which is adjustable between an open position where the access sites are exposed and a closed position where the access sites are enclosed within the chamber.

The device may also include a controller, e.g., a microprocessor or other suitable computing device, which actuates the UV light source within the chamber to illuminate the UV light source based on various conditions. For example, in response to an adjustment of the enclosure from the open position to the closed position wherein the access sites are enclosed within the chamber of the enclosure, the ultraviolet light source may be illuminated for a predetermined first duration while the enclosure is maintained in the closed position. Following illumination of the ultraviolet light source for the first duration, the ultraviolet light source may then be illuminated for a predetermined second duration at predetermined time intervals while the enclosure is maintained in the closed position. In this way, following an initial disinfection with UV light, disinfection of the access sites may be maintained by periodically cycling UV light treatment of the access sites while the access sites remain enclosed within the chamber.

In some examples, a notification system may be included with the device. The notification system may provide indications of various operating conditions of the device. For example, the notification system may provide visual signals indicating when the UV light is illuminated and/or indicating a disinfection state of the access ports enclosed within the chamber of the enclosure. For example, the notification system may include an alarm and/or locking mechanism to warn a user that the device has been opened while the access sites are not in “non-infectious status,” e.g., before a full UV dose has been delivered to adequately disinfect or sterilize the access sites.

In some examples, the durations and/or intensities of the UV light used to disinfect the access sites enclosed within the chamber may be adjusted based on various physical attributes of the device (e.g., the geometry of the chamber within which the access sites are contained), the type of access sites being sanitized, and/or based on a calibration of the device for a particular application in a given therapeutic setting. For example, the durations and/or intensities of the UV light may be adjusted based on data obtained from a look-up table. The data in the look-up table may be obtained from models based on experimental data which correlates UV dosing parameters with physical attributes of the device and the type of access sites included in the device. Additionally, microbiologic data which describes microbe-specific dosing and timing of UV light may be used to adjust the durations and/or intensities of the UV light to ensure adequate sanitization while using a minimal amount of UV light. In this way, non-infectiousness of access sites may be guaranteed whenever the access sites are accessed while potentially reducing power consumption of the device.

In some examples, some components of the device may be packaged in a modular fashion. For example, the device may comprise a precisely defined, two part body or housing which constrains the geometry of the access sites or ports (of potentially many different port-containing devices). For example, a transmitting portion or module may contain a controller, a UV source, a battery, and may be reusable and interchangeable with different receiving portions or modules which incorporate, house, or otherwise attached to ports of intravenous or other medical devices with access sites of potential infection risk. In this example, the controller in the transmitting module may be used to illuminate the UV light source using a minimal amount of energy sufficient to adequately disinfect access ports in a particular receiving module to which the transmitting module is coupled. In this way, power consumption of the device may be reduced, e.g., battery life may be extended, and dimensional constraints of the device may be reduced so that the device may be used in a portable fashion, for example.

Such an approach enables automated disinfection of access sites by ensuring that the access sites are clean of microbial contamination before each use. Automatically maintaining access site disinfection in this way may lead to a reduction or prevention of infections during surgeries and procedures involving medical devices with access sites which are prone to contamination. Because such an approach is automated, practitioner hardships and errors associated with repeatedly performing arduous tasks every time a port is accessed may be reduced thereby potentially freeing up practitioner availability for other tasks while reducing healthcare costs associated with infection occurrences in patients.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 schematically shows a device for maintaining disinfection of access sites of medical devices in accordance with the disclosure.

FIGS. 2-9 show various example embodiments of a device for maintaining disinfection of access sites of medical devices in accordance with the disclosure.

FIG. 10 shows a graph of bacterial colony counts versus ultraviolet light exposure.

FIG. 11 shows an example method for maintaining disinfection of access sites of medical devices in accordance with the disclosure.

FIG. 12 shows example graphs illustrating a method for maintaining disinfection of access sites of medical devices in accordance with the disclosure.

FIGS. 13-26 show various additional example embodiments of a device for maintaining disinfection of access sites of medical devices in accordance with the disclosure.

FIG. 27 schematically shows an example computing device in accordance with various embodiments.

### DETAILED DESCRIPTION

Embodiments described herein are directed to devices and methods for automatically maintaining disinfection of access sites of medical devices, such as ports/hubs of central venous catheters, dialysis catheters, or other medical tubing, using an ultraviolet (UV) light source which is selectively illuminated during various conditions in order keep the access sites sterile and ready for use. As used herein, the term “access site” may refer to any element of a medical device which is potentially exposed to contamination, e.g., injection ports of catheters or skin entry points of catheters in a patient. Further, the terms “access site,” “port,” “hub,” and “entry point” as used herein are synonymous.

The term “disinfected” as used herein refers to bacterial colony counts being reduced below a threshold level. For example, as used herein, an access site which is disinfected may refer to an access site which has had 50%, 60%, 80%, 90%, or 99% of the bacteria on the access site destroyed or removed. Further, the term “non-disinfected state” as used herein may refer to bacterial colony counts remaining greater than a threshold level. Further, indicating a non-disinfection state as used herein may refer to indicating an amount or percentage of bacteria on the access site which has been destroyed or removed. The term “sufficiently disinfected” when used in reference to an access site may refer to an access site which has had at least a predetermined threshold amount of bacteria on the access site destroyed. In some examples, the term “sterilized” as used herein may refer an access site which has had substantially all of the bacteria on the access site destroyed or removed.

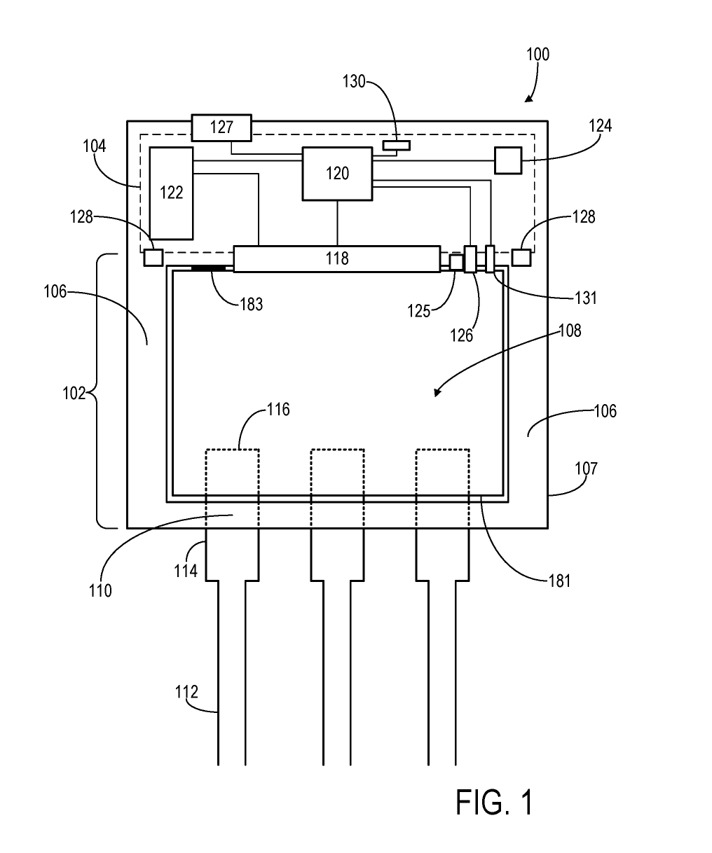

Turning to the figures, FIG. 1 schematically shows a device 100 which may be used to automatically maintain one or more access sites of medical devices in a sufficiently disinfected or sterilized state. Device 100 includes an enclosure 107 which is structured to receive and enclose access sites of medical devices, e.g., access site 116 at an end 114 of a medical device 112, within a chamber 108 formed in or defined by the enclosure 107. Device 100 includes a UV light source 118 which directs UV light into the chamber 108 and onto the access sites for preventing and treating contamination of the access sites. For example, access site 116 may be a port or hub in an end of tubing of a catheter. As another example, access site 116 may be a skin entry point of a catheter in a patient. The enclosure may be configured to receive and contain any number of access sites, e.g., a single access site or a plurality of access sites, via apertures 110 in the enclosure. By way of example, three different access sites are shown enclosed within device 100 in FIG. 1. In some examples, after installation around access sites of medical devices interfacing with a patient, device 100 may remain external to the patient and may be left on the access sites allowing essentially continuous assurance of port disinfection via scheduled actuation of the UV light source 118 controlled by a controller 120 included in device 100.

Device 100 comprises a plurality of different components which may be included within a single body or housing. In some examples, some components of the device may be packaged or grouped together in a modular fashion. For example, the device may comprise a precisely defined, two part box which constrains the geometry of the access sites or ports (of potentially many different port-containing devices). For example, a transmitting portion or transmitting module 104 may contain a controller 120, a UV source 118, a power source 122 such as a battery, and other components (examples of which are described below). The transmitting module 104 may be self-contained, reusable, and interchangeable with different receiving portions or receiving modules which incorporate, house, or otherwise attached to ports of intravenous or other medical devices with access ports of potential infection risk. In this example, the controller 120 in the transmitting module 104 may be used to illuminate the UV light source 118 using a minimal amount of energy sufficient to adequately disinfect access ports in a particular receiving module to which the transmitting module is coupled.

In some examples, the transmitting module 104 and the receiving module 102 may include complementary mating features 128 so that the transmitting module may be releasably coupled to the receiving module such that the ultraviolet light source in the transmitting module is configured to direct ultraviolet light into a chamber 108 of an enclosure 107 of the receiving module 102. For example, different receiving modules with different geometries (e.g., physical dimensions and shapes) may include docking elements for receiving the transmitting module 104. For example, the transmitting module 104 may be removed from a first receiving module and installed in a second, different receiving module.

In order to calibrate operational features of the transmitting module to perform optimally for a specific configuration of a receiving module to which it is attached or within which it is installed, a detecting device 131 may be included in the transmitting module 104 for detecting the receiving portion. For example, the detecting device 131 may include one or more sensors configured to detect geometric dimensions of the chamber of the receiving module so that various operational features, such as UV light dose durations and UV light intensities, may be adjusted. In this way, the detection device may be used to recognize and identify the receiving module in order to determine the type and number of access sites contained in the receiving module and which UV light algorithm/regimen should be used during disinfection maintenance. For example, a central line may most likely be infected by staphylococcus aureus (staph), thus a staph killing UV dose and timing may be used. As another example, if the device is attached to a urinary catheter, the contaminating organism may be escherichia coli (e. coli), therefore a different UV dose and timing may be used. In some examples, user input may be used to calibrate operational features of the transmitting module after it is installed into a receiving module. For example, a user may input a receiving module type, size, volume, shape or other physical parameters associated with the receiving module into a user interface included in the transmitting module so that operating parameters of the transmitting module can be tailored to the specific receiving module to which it is attached.

The receiving module 102 comprises an enclosure 107 configured to engage an end 114 of a medical device 112 such that an access site 116 of the medical device 112 is positioned within a chamber 108 of the enclosure. The chamber 108 may be defined by enclosure walls 106 which form a reservoir around installed access ports. The chamber may have any suitable geometry, e.g., any suitable length, width, depth, and shape (examples of which are described herein). For example, the enclosure walls 106 may include two pairs of opposing side walls extending upwardly from a bottom surface to form an enclave within the enclosure walls.

The enclosure may include a sealing member 181, e.g., a door, flap, or other moveable member, which is coupled to device 100 via a moveable coupling element 183, e.g., one or more hinges or similar features, so that the sealing member is adjustable between a first and second position. For example, in the first position the sealing member may expose the interior of chamber 108 and any access ports contained therein, and in the second position the sealing member may cover the chamber 108 so that any access ports in the chamber are enclosed within the chamber and cannot be accessed unless the sealing member is opened. By adjusting the sealing member to the second position, the enclosure is placed in a closed position. In the closed position, the enclosure 107 is configured to enclose the access sites, e.g., access site 116, within the chamber 108. By adjusting the sealing member to the first position, the enclosure is placed in an open position. In the open position the enclosure is configured to expose the access sites, e.g., access site 116. In the closed position, a complete and uninterrupted light-tight seal may be formed preventing light from escaping out of the enclosure.

For example, the enclosure 107 may be closed around the injection hubs of a multi-lumen central venous catheter or the entry points or a dialysis catheter. When closed, the enclosure does not occlude or compress the catheter lumens but encloses the injection hubs completely within chamber 108. The enclosure may fit over an IV port or central line ports and may be configured to accept injection hubs and hold the hubs in a position within chamber 108 which is in a path of maximal light from the UV light source 118. As described in more detail below, when the enclosure is closed around the access sites, the UV light source may be illuminated to kill or disable bacteria on the access sites with UV light. After a predetermined time duration has elapsed, where the predetermined time duration may depend on a strength (e.g., a wavelength and/or intensity) of the UV light source, the UV light source may be turned off. In some embodiments, to keep bacterial counts low, the UV light may be cycled on again periodically if the enclosure is not opened.

In some examples, the enclosure walls surrounding the chamber may be composed of an opaque or semi-opaque material, e.g., a plastic or other suitable UV light shielding material so that when the UV light source is illuminated while the enclosure is in the closed position, substantially no UV light will exit the chamber during the disinfection process. Further, in some examples, a least a portion of the interior walls of the enclosure, e.g., the interior walls defining chamber 108, may include a UV-reflective coating, e.g., a mirror coating, in order to increase an amount of UV light directed to the access sites.

As remarked above, the transmitting module 104 includes an ultraviolet light source 118. When the transmitting module 104 is installed within, coupled to, or mated with a receiving module, the UV light source is positioned to specifically direct light onto the access sites within the chamber and may not transmit light onto portions of the medical device outside of the chamber. For example, UV light may be constrained by the device so that UV light is not transmitted through the tube lumen interior of a catheter beyond the access site of the catheter.

The UV light source 118 may comprise one or more of any suitable UV light sources. For example, the UV light source may comprise one or more ultraviolet light-emitting diodes (LEDs). Other nonlimiting example UV light sources include mercury lamps, black lights, short wave ultraviolet lamps, ultraviolet lasers, gas discharge lamps, etc. The frequency/wavelength of light emitted from the UV light source may be any suitable frequency/wavelength within the UV range such as wavelengths in a range between 400 nm and 10 nm, e.g., a wavelength of 260 nm may be used. In some examples, the frequency/wavelength and/or intensity of the UV light may be adjusted based on a geometry of the chamber 108, the types of access sites included in the chamber, a state of charge of a battery included in the device, etc. Further, the number of UV sources used and the types of UV sources actuated in the device may depend on a particular application or a specific geometry of the receiver portion in which the transmitting portion is included. In some embodiments, the wavelength of the UV light may be selected based on an identified optimal germicidal effectiveness and/or absorbances of germicidal moieties such as cytosine, adenine, guanine, thymine, uracil, etc. For example, a low pressure UV light may be configured to emit UV light having a wavelength in an approximate range of 240-260 nm, e.g., 253.7 nm, and a medium pressure UV light may be configured to emit UV light having a wavelength in an approximate range of 300-320 nm.

The power source 122 included in the transmitting module 104 may comprise any suitable power supply. For example, the power source may comprise one or more batteries, e.g., rechargeable batteries such as lead-acid, nickel cadmium (NiCd), nickel metal hydride (NiMH), lithium ion (Li-ion), or lithium ion polymer (Li-ion polymer) batteries. As another example, power source 122 may be coupled to an external power supply such as an external outlet, generator, or battery. In some embodiments, one or more Universal Serial Bus (USB) ports may be included in the device so that, when attached to a USB port in the device, a USB cord may supply power to the device, e.g., to recharge one or more batteries in the device. As another example, when attached to a USB port in the device, a USB cord may be used to transmit data to and/or from an external computing device or system.

In some examples, the transmitting module 104 may include a switch 125 in communication with controller 120. The switch may be actuated when the enclosure 107 is adjusted from the closed position to the open position or from the open position to the closed position. For example, switch 125 may interface with sealing member 181 and may be configured to be actuated in response to a change in position of the sealing member between the first position and the second position to determine if the device is opened or closed. As another example, switch 125 may comprise an actuating mechanism that may be actuated by a user of the device to initiate UV disinfection and/or disable UV disinfection of any access sites included in the device.

In some examples, device 100 may include a locking mechanism 126. The locking mechanism 126 may interface with sealing member 181 to lock the sealing member in a closed position during certain conditions. For example, the locking mechanism may comprise a latch which is configured to engage with a portion of the sealing member in response to a signal received from controller 120. For example, the locking mechanism 126 may be configured to prevent adjustment of the enclosure from the closed position to the open position and the controller may be configured to lock the enclosure in the closed position via the locking mechanism when the ultraviolet light source is illuminated and/or when the access sites in the chamber are in a non-disinfected state. By preventing access to the chamber during certain conditions, e.g., if the UV light is powered on and/or if the access sites are in a non-disinfected state, the locking mechanism may prevent user exposure to UV light and prevent use of a contaminated access site until a disinfection cycle is complete.

In some examples, the transmitting portion may further include a locking override mechanism 127 which may be used to override or disengage the locking mechanism 126 during certain conditions. For example, in response to an actuation of the locking override mechanism while the enclosure is in the closed position and the ultraviolet light source is illuminated, illumination of the ultraviolet light source may be discontinued and the locking mechanism may be unlocked to permit adjustment of the enclosure from the closed position to the open position. The locking mechanism may comprise a manually actuated button or switch, a speech input device, or other suitable user input device which may be actuated by a user to bypass the locking mechanism in order to open the device, e.g., in cases of emergency.

In some examples, the transmitting portion may further include a notification system 124. Notification system 124 may include one or more indicator elements such as display devices, lights (e.g., LED lights), audio speakers, haptic devices, etc. For example, notification system 124 may be used to indicate various operational states of the device, e.g., whether the UV light is on or off, whether the enclosure is in the open or closed state, whether the access ports are sufficiently disinfected or not, etc. For example, the notification system may include a light which may be illuminated in response to certain conditions, or a color of the light may be changed to indicate a change in the operational state of the device, or speakers in the notification system may emit sounds or vibrations in response to a change in operational state. As an example, the notification system may be used to provide an alarm to warn a user that the device has been opened while ports are not in “non-infectious status,” e.g., before a full UV dose has been delivered to achieve sufficient disinfection. As another example, an indication may be provided to the user by the notification system via a visual signal when an access port in the chamber of the enclosure may be safely used because a disinfection cycle is complete.

In some examples, usage data of device 100 may be recorded and/or broadcast to an external computing device. Thus, the transmitting module 104 may include a broadcasting device 130 which is configured to send device usage data to a remote computing device over a network. Nonlimiting examples of broadcast devices include antennas, modems, radio transmitters, ethernet ports, USB ports, etc. For example, for each adjustment of the enclosure from the closed position to the open position, an access timestamp may be associated with said adjustment and stored in a data-holding system in the transmitting module and, if said adjustment is performed while the access sites are in a non-disinfected state, a flag may be associated with the access timestamp and stored in the data-holding subsystem. This usage data (the access time-stamps and flags) may then be sent to a remote computing device via the broadcasting device 130. In this way, usage data may be used to generate reports for medical personnel or supervisors for compliance evaluations or other applications. For example, the usage data may include information relating to how often a port was accessed during a given time period and if and how often a port was accessed while the port was in a non-disinfected or potentially contaminated state.

The controller 120 may comprise any suitable computing device. In some embodiments, the controller may comprise physical circuitry, e.g., physical circuitry on a printed circuit board (PCB), programmed to perform one or more of the various acts described herein. For example, controller 120 may comprise a small microprocessor with a timing circuit which controls the on-off cycling of the UV source such that every time the device is closed the UV light is switched on for a first duration and then periodically switched on in order to maintain disinfection of the access sites. The UV light on/off cycling may be timed to ensure that the ports are sterile whenever they are accessed. For example, the enclosure may be placed on the ports of a catheter at the time of catheter line placement in a patient and opened whenever it is necessary to access the line. Following access, the enclosure may be re-closed and the ultraviolet light cycling resumed. While the enclosure is open, the light may be prevented from illuminating, thus shielding the patient and provider. The controller may include a logic subsystem and a data-holding subsystem comprising machine-readable instructions stored thereon that are executable by the logic subsystem to perform various disinfection maintenance methods, such as the example method 1000 described below. The routines and instructions described herein may represent code stored in the controller and may be carried out by the controller in cooperation with one or more hardware elements, including sensors, actuators, etc. In this way, example methods described herein may be carried out by the controller operating in combination with one or more hardware elements, such as the noted sensors, actuators, etc.

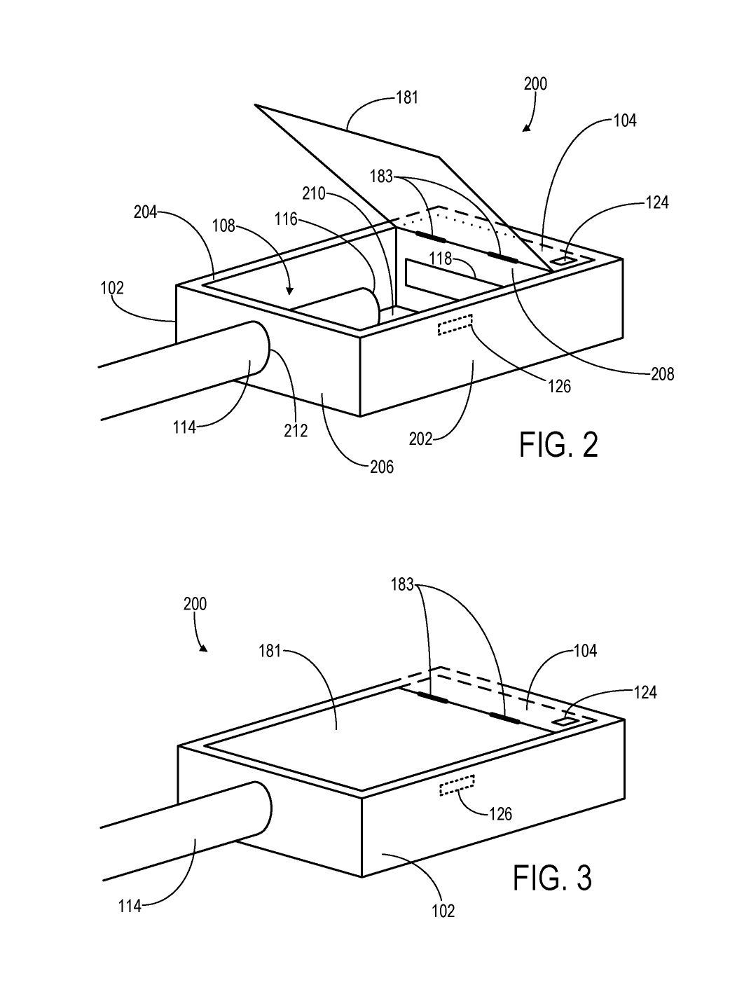

FIGS. 2-9 show various example embodiments of a device for maintaining disinfection of access ports of medical devices. Like-numbered elements appearing in FIGS. 2-9 correspond to like-numbered elements shown in FIG. 1 described above. It is to be understood that these embodiments are exemplary in nature and are not to be considered in a limiting sense, because numerous variations are possible.

FIGS. 2 and 3 show an example embodiment 200 of device 100 with a receiving module 102 which includes an enclosure 107 which takes of the form of a box with a chamber 108 defined by first and second opposing lateral sides 202 and 204 coupled to first and second opposing longitudinal sides 206 and 208, where the sides 202, 204, 206, and 208 extending upwardly from a bottom side 210. In this example, an aperture 212 is included in side 206 of the box and is sized to receive an end 114 of medical tubing such that an access site 116 of the tubing is contained within the chamber 108 of the enclosure. In some examples, aperture 212 may engage with features located at end 114 adjacent to access site 116 of the medical tubing so that the access site may be releasably held within chamber 108. For example, after a catheter line is placed in a patient, a hub of the catheter may be inserted into aperture 212 so that the hub remains in place within the chamber while the catheter remains in the patient.

The transmitting module 104 is included within the body or housing of the receiving module 102 such that the UV light from the UV light source 118 is directed into the chamber 108. A door 181 is coupled to a top portion of wall 208 via hinges 216 to permit the adjustment of the door between a first position placing the enclosure in the open position as shown in FIG. 2, and a second position placing the enclosure in the closed position as shown in FIG. 3. As shown in FIG. 2, in the open position the door is in a position which exposes the chamber 108 and the access site 116 so that the access site 116 may be accessed to administer fluids to a patient, for example. In the closed position shown in FIG. 3, edges of the door 181 interface with the side walls 202, 206, 204, and 208 of the enclosure to substantially seal off the chamber so that UV light does not escape the chamber during the disinfection maintenance procedure. After the device is installed around the access site, the device may remain in the closed position while coupled to the access site so that disinfection maintenance is continuously performed until access to the access site is needed.

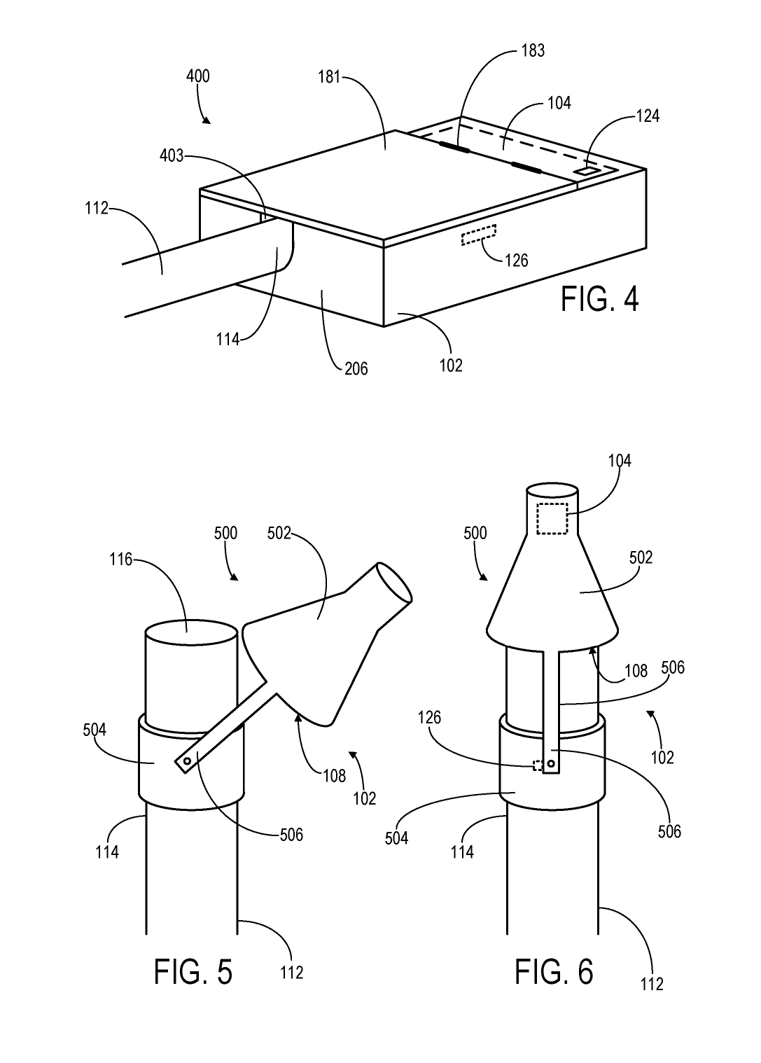

The access site may be held within the chamber 108 of the device in any suitable way. As another example, FIG. 4 shows another embodiment 400 of a device for maintaining disinfection of access ports of medical devices. Embodiment 400 is similar to the embodiment 200 shown in FIGS. 2 and 3. The device is shown in the closed position in FIG. 4. In this example, a cut-out 403 is included in side wall 206 of the enclosure such that when the door is closed over the chamber, the door 181 positions and holds the end 114 of the medical tubing in place so that the access site is held within the chamber of the enclosure. In this example, when the door is adjusted to the open position exposing the chamber and the access site, the end of the tubing is disengaged so that a user can access the access site or remove the access site from the chamber.

FIGS. 5 and 6 show another example embodiment 500 of a device for maintaining disinfection of access ports. In this example, the receiving module 102 includes an enclosure 502 which takes the form of a cap defining a chamber 108 within the enclosure 502. The cap is coupled to an end 114 of a medical tube adjacent to an access site 116 of the tube via a hinged element 506 coupled to a support element 504 wrapped around the tube. In this example, the support element 504 is a cylindrical brace which may be slipped onto the end 114 of the tubing to hold the receiving module 102 in place. Though the enclosure 502 is shown with a conical shape in FIGS. 5 and 6, the enclosure 502 may be any suitable shape, e.g., cylindrical, box-shaped, etc. The enclosure 502 is adjustable between an open position (shown in FIG. 5) wherein the access site is exposed and a closed position (FIG. 6) wherein the access site is contained within the chamber 108 of the enclosure. The transmitting module 104 may be included in the enclosure at any suitable location, e.g., near an apex or distal end of the enclosure 502 as shown in FIG. 6. A locking mechanism 126 may be included adjacent to the hinged element 506 to lock the hinged element 506 in place while the enclosure is in the closed position when light is directed from the UV source in the transmitting module 104 onto the access site 116 contained in the chamber 108.

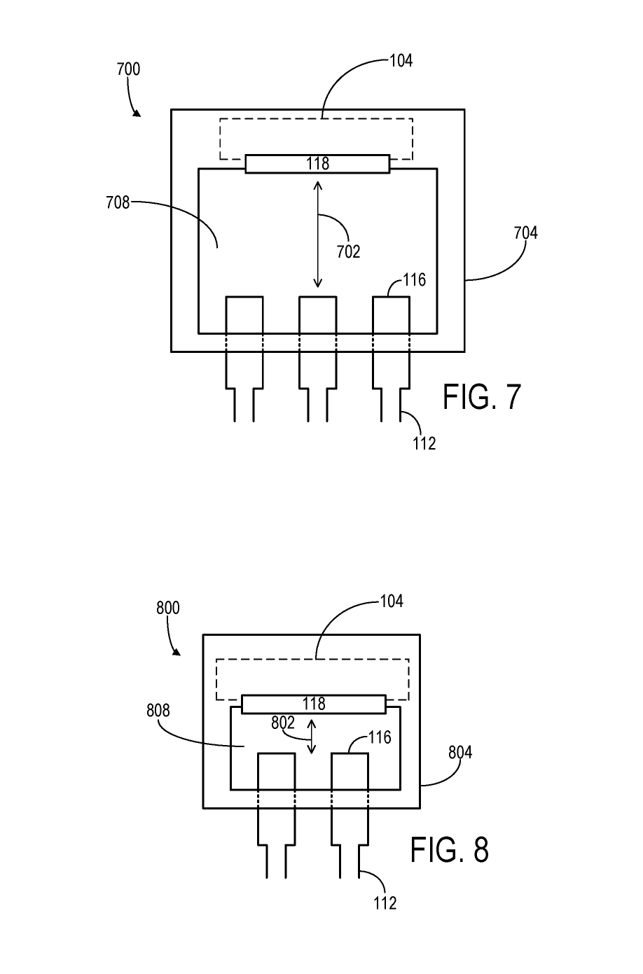

As remarked above, in some examples, some components of the device may be packaged or grouped together in a modular fashion. For example, the transmitting module 104 may be self-contained, reusable, and interchangeable with different receiving modules which incorporate, house, or otherwise attached to ports of intravenous or other medical devices with access ports of potential infection risk. For example, FIG. 7 shows a transmitting module 104 installed within a first receiving module 704 which has a chamber 708 wherein the access sites are positioned a first distance 702 from the UV light source 118 of the transmitting module 104. FIG. 8 shows the same transmitting module 104 installed within a second receiving module 804 which has a chamber 808 wherein the access sites are positioned a second distance 802 from the UV light source 118 of the transmitting module 104.

In order to calibrate operational features of the transmitting module 104 to accommodate the different configurations of the different receiving modules 704 and 708, a calibration may be performed in the transmitting module 104 in order to adjust an intensity of the UV light emitted from the light source and/or a duration of illumination of the UV light source to achieve adequate disinfection of the ports. For example, since distance 702 in chamber 708 of device 700 is greater than distance 802 in chamber 808 of device 800, an intensity and/or duration of UV illumination used in device 700 may be greater than an intensity and/or duration of UV illumination used in device 800.

Further, an intensity and/or duration of UV illumination used by the transmitting module may also depend on the type of access ports enclosed in the receiving module. In some examples, the type of access ports enclosed in the receiving module may be determined based on an identification of the receiving module, e.g., via a detection device or user input as described above. The calibration of operating parameters of the transmitting module may be based on data obtained from a lookup table, e.g., a lookup table stored in a memory component in the device. For example, given a specific geometry, configuration, or application of a particular receiving module, corresponding UV dosing information contained in a lookup table may be loaded into the controller to control the UV light source. The data in the lookup table may be based on models obtained from results of experiments which correlate UV dosing parameters with a given UV light source for particular access ports in particular receiving module configurations in order to obtain adequate disinfection.

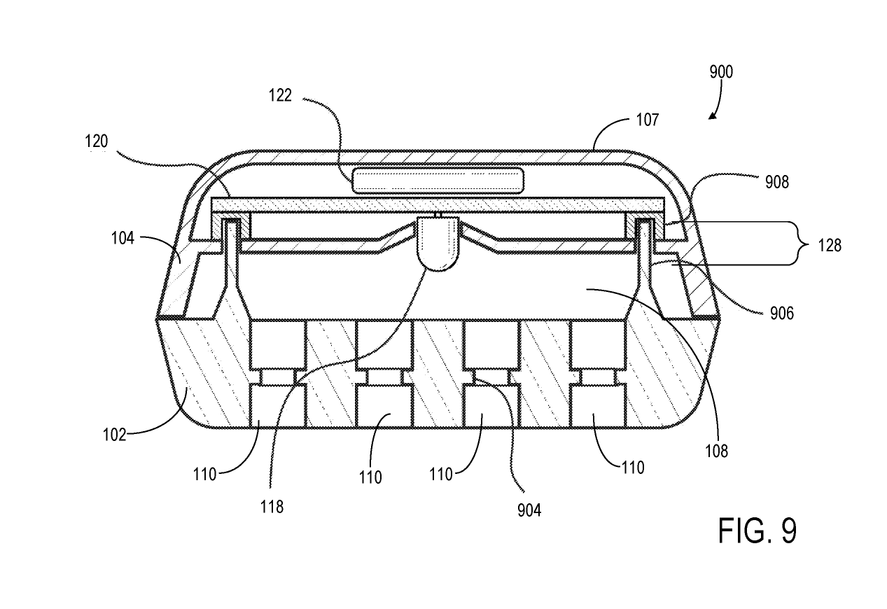

FIG. 9 shows another example embodiment 900 of a device for maintaining disinfection of access sites of medical devices. FIG. 9 is drawn approximately to scale. FIG. 9 shows a transmitting module 104 which includes a light source 118, e.g., one or more LEDs, coupled to a controller 120 in the form of a printed circuit board, and a battery 122. The transmitting module 104 is removably coupled to a receiving module 102 to form a chamber 108. The transmitting module includes apertures 110 configured to receive access sites of medical devices. In this example, the apertures include keying features 904 which may be used to repeatedly locate each access site tip with the option of leaving one or more tips outside of the enclosure if desired. The transmitting module 104 and the receiving module 102 include complementary mating features 128 comprising tabs 906 in the receiving module 102 and optical interrupter slots 908 mounted on the printed circuit board in the transmitting module 104 which obstruct light. The tabs 906 in the receiving module may uniquely identify the receiving module type so that the disinfections algorithms may be adjusted accordingly as described below.

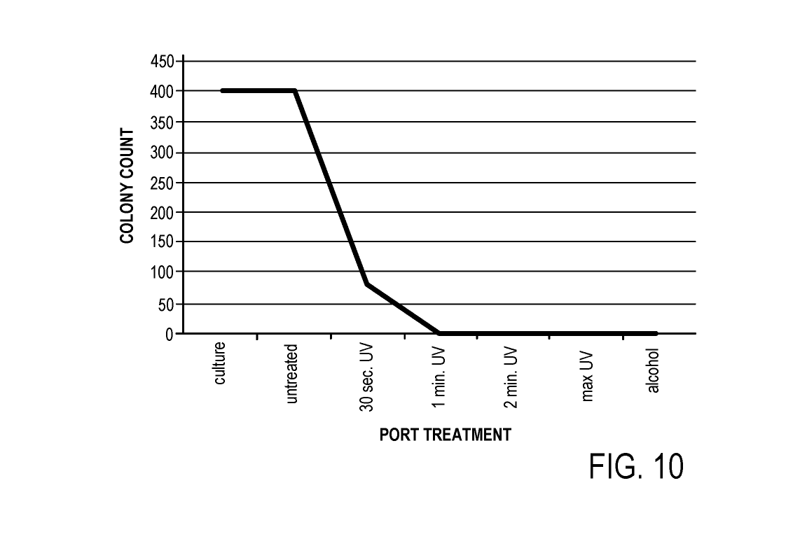

FIG. 10 shows a graph of example data which may be used to calibrate operating parameters of a transmitting module based on the type of contamination expected to occur on a particular access site. In particular, FIG. 10 shows bacterial colony counts on an access port versus ultraviolet light exposure after staphylococcus aureus inoculation. In this model, the device was highly bactericidal, with less than one minute of exposure required to reduce bacterial counts to zero. The graph also shows a comparison with the current clinical standard procedure, which is a 5 second scrub with an alcohol wipe.

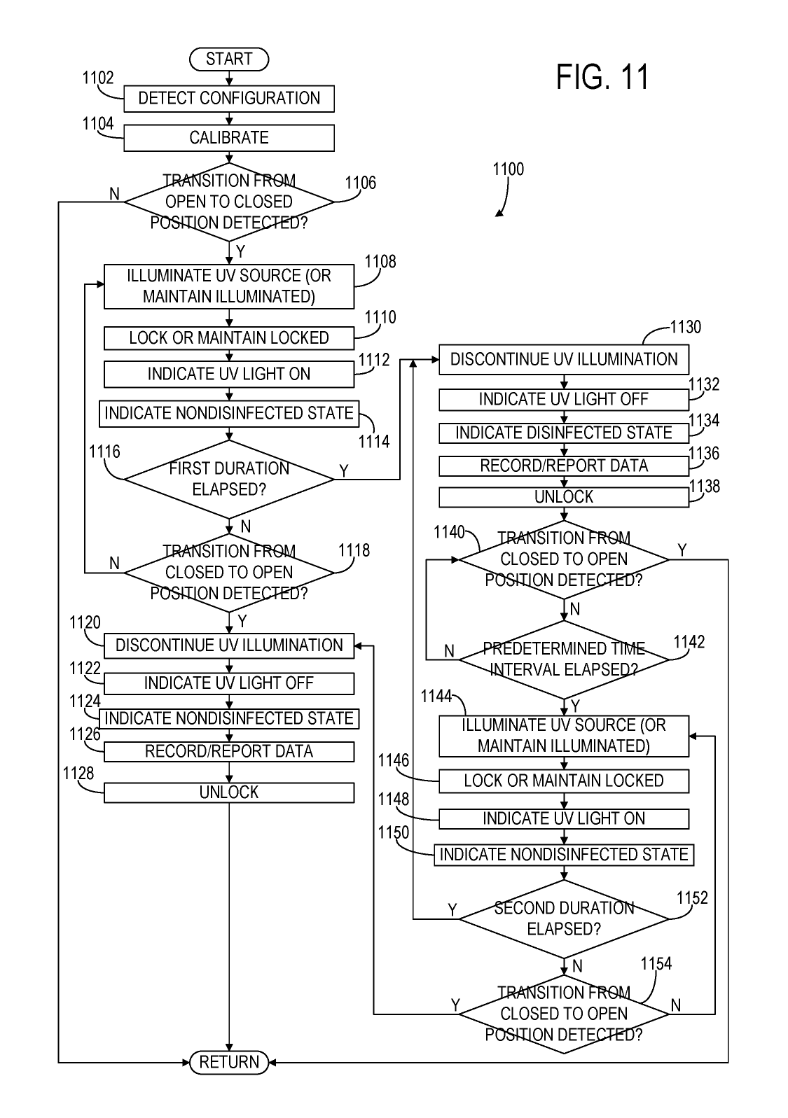

FIG. 11 shows an example method 1100 for maintaining disinfection of access sites of medical devices using a device, such as device 100 described above, with an ultraviolet (UV) light source in an enclosure which is selectively illuminated during various conditions in order keep the access sites in the enclosure sterile and ready for use. It should be understood that the steps shown in FIG. 11 may be performed in the sequence illustrated, in other sequences, in parallel, or in some cases omitted. For example, one or more steps of method 1100 may be optional and thus omitted and the steps may be performed in any suitable order.

At 1102, method 1100 may include detecting a configuration of the device. For example, detecting device 131 may be used to detect geometric dimensions of a chamber of a receiving module within which a transmitting module is installed so that various operational features, e.g., UV light dose durations, UV light maintenance scheduling, and/or UV light intensities, may be adjusted. As remarked above, the detection device may be used to recognize and identify the receiving module and, therefore what kind of port and which UV light algorithm/regimen should be used during disinfection maintenance. In some examples, user input may be provided to the device via a suitable user input device. For example, a user may input a receiving module type, size, volume, shape or other physical parameters associated with the receiving module into a user interface included in the transmitting module so that operating parameters of the transmitting module can be tailored to the specific receiving module to which it is attached.

At 1104, method 1100 may include calibrating the device. For example, based on the detected configuration of the device, e.g., the detected geometric dimensions of a chamber in an enclosure of the receiving module, operational parameters of the transmitting module may be calibrated. As remarked above, the calibration of operating parameters of the transmitting module may be based on data obtained from a lookup table stored in a memory component in the device. For example, given a specific geometry, configuration, or application of a particular receiving module, corresponding UV dosing information such as UV intensity, UV duration, and UV illumination scheduling may be loaded into the controller to control the UV light source. The data in the lookup table may be based on models obtained from results of experiments which correlate UV dosing parameters with a given UV light source for particular access ports in particular receiving module configurations in order to obtain adequate disinfection. Examples of UV dosing information which may be calibrated include a first duration of UV light illumination which is used after an initial closure of the device around access sites and a second time duration of UV illumination which is used to periodically maintain disinfection of the access sites while the device remains closed around the access sites. In some examples, this second duration of illumination used to maintain disinfection may be shorter than the first duration used to perform an initial disinfection following closure of the device around the access ports.

At 1106, method 1100 includes determining if a transition from an open position to a closed position is detected. For example, switch 125 may be used to monitor adjustments of sealing member 181 from the open position to the closed position to determine when the device is closed around access ports. If a transition from the open position to the closed position is not detected at 1106, method 1100 may end or may return to continue monitoring the device to detect changes from the open to closed state or to detect other configuration changes of the device. However, if a transition from an open position to a closed position is detected at 1106, method 1100 proceeds to 1108.

At 1108, method 1100 includes illuminating the UV source. For example, the controller 120 may send a signal to the UV light source to cause the UV light source to become illuminated for a predetermined first duration while the enclosure is maintained in the closed position. The predetermined first duration may be obtained from a look-up table and may be based on configuration information associated with the device or access sites contained within the device. In this way, after the device is closed around access sites an initial UV dose may be applied to the access ports for the first duration in order to sufficiently sanitize the access sites.

At 1110, method 1100 may include locking the device. For example, the controller 120 may send a signal to locking mechanism 126 to cause the locking mechanism to engage with the sealing member 181 so as to lock the enclosure in the closed position while the ultraviolet light source is illuminated. In this way, the locking mechanism 126 may prevent adjustment of the enclosure from the closed position to the open position while the ultraviolet light source is illuminated to sanitize the access sites.

At 1112, method 1100 may include providing an indication that the UV light is on. For example, controller 120 may send a signal to the notification system 124 to actuate one or more indicators in the notification system to provide an indication that the ultraviolet light is illuminated. At 1114, method 1100 may include providing an indication that the access sites within the device is in a non-disinfected state. For example, controller 120 may send a signal to the notification system 124 to actuate one or more indicators in the notification system to provide an indication that the first duration has not yet elapsed and thus the access sites may not yet be in a sufficiently disinfected or sterilized state. As remarked above, the notification system may include various indicators such as lights, speakers, haptic devices, etc. which may be actuated by the controller to provide visual, audible, and/or haptic indications of operational states of the device.

At 1116, method 1100 includes determining if the first duration has elapsed. For example, the controller 120 may include a timing circuit which determines when/if the first duration of UV illumination has elapsed following the initial closure of the device around the access sites. If the first duration has not elapsed at 1116, method 1100 proceeds to 1118 to determine if a transition from the closed position to the open position is detected. For example, switch 125 may be used to monitor adjustments of sealing member 181 from the closed position to the open position to determine if an adjustment of the device from the closed position to the open position is initiated. In some examples, while the UV light is illuminated the device may be maintained locked, e.g., via locking mechanism 126, in order to prevent access to the access sites before the access sites are sufficiently disinfected or to prevent user exposure to UV light. Thus, in some examples, determining if a transition from the closed position to the open position is detected may include determining if a locking override mechanism is actuated. For example, a user may provide input to actuate the locking override mechanism 127 in order to bypass the locking mechanism so that the device may be opened. As described below, in response to an actuation of the locking override mechanism while the enclosure is in the closed position and the of a medical device such that an access site of the medical device is positioned within a chamber tinued and the locking mechanism may be unlocked to permit adjustment of the enclosure from the closed position to the open position.

If a transition from the closed position to the open position is not detected at 1118, method 1100 proceeds back to 1108 to continue illuminating the UV source for the first duration to disinfect the access sites. However, if a transition from the closed position to the open position is detected at 1118, then method 1100 proceeds to 1120. At 1120, method 1100 includes discontinuing UV illumination. For example, the controller may be configured to discontinue illumination of the ultraviolet light source in response to an adjustment of the enclosure from the closed position to the open position so that exposure of a user to UV light is prevented when the device is opened.

At 1122, method 1100 may include providing an indication that the UV light is off. For example, controller 120 may send a signal to the notification system 124 to actuate one or more indicators in the notification system to provide an indication that the ultraviolet light is not illuminated. At 1124, method 1100 may include providing an indication that the access sites within the device are in a non-disinfected state since the UV disinfection dose was terminated before adequate disinfection or maintenance of disinfection is complete.

At 1126, method 1100 may include recording and/or reporting device usage data. As remarked above, in some examples, usage data of device 100 may be recorded and/or broadcast to an external computing device. Thus, the transmitting module 104 may include a broadcasting device 130 which is configured to send device usage data to a remote computing device over a network. For example, for each adjustment of the enclosure from the closed position to the open position, an access timestamp may be associated with said adjustment and stored in a data-holding system in the transmitting module and, if said adjustment is performed while the access sites are in a non-disinfected state, a flag may be associated with the access timestamp and stored in the data-holding subsystem. This usage data (the access time-stamps and flags) may then be sent to a remote computing device via the broadcasting device 130.

At 1128, method 1100 may include unlocking the device. For example, the controller 120 may send a signal to locking mechanism 126 to cause the locking mechanism to disengage with the sealing member 181, e.g., by deactuating the locking mechanism, so as to unlock the enclosure after illumination of the ultraviolet light source is terminated so that the device may be opened and the access sites accessed by a user.

Returning to 1116, if the first duration of UV illumination used to initially disinfect the access ports following closure of the access sites within the device has elapsed, then method 1100 proceeds to 1130. At 1130, method 1100 includes discontinuing UV illumination, and, at 1132, method 1100 may include providing an indication, e.g., via the notification system 124, that the UV light is off. At 1134, method 1100 may include providing an indication, e.g., via the notification system 124, that the access sites within the device are in a sufficiently disinfected state. At 1136, method 1100 may include recording and/or reporting usage data, and, at 1138, method 1100 may include unlocking the device so that device may be opened by a user to access the disinfected access ports.

At 1140, method 1100 includes determining if a transition from the closed position to the open position is detected. For example, a user may open the unlocked device in order to access the access ports to administer fluids or perform other operations using the disinfected access sites. If a transition from the closed position to the open position is detected at 1140, then method 1100 may end or return to start. However, if a transition from the closed position to the open position is not detected at 1140, e.g., if the device remains in the closed position following the initial UV disinfection for the first duration, then UV light may be used to maintain disinfection of the access sites while the device remains closed around the access sites and thus method 1100 proceeds to 1142.

At 1142, method 1100 includes determining if a predetermined time interval has elapsed. For example, after the initial disinfection of the access sites for the first duration, the UV light source may be illuminated at predetermined time intervals while the device remains closed around the access ports in order to maintain sufficient disinfection of the access ports. The predetermined time interval may be based on configuration information of the device detected in step 1102 described above and may be adjusted based on this configuration information. For example, if the transmitting module is coupled with a first receiving module, a first predetermined time interval may be used to schedule UV disinfection maintenance, whereas if the transmitting module is coupled with a second receiving module, a second, different predetermined time interval may be used to schedule UV disinfection maintenance.

If the predetermined time interval has not elapsed at 1142, then method 1100 proceeds back to 1140 to continue monitoring the device to detect a transition from the closed position to the open position while waiting until the predetermined time interval has elapsed. If the predetermined time interval has elapsed at 1142, then method 1100 proceeds to 1144.

At 1144, method 1100 includes illuminating the UV source. For example, the controller 120 may send a signal to the UV light source to cause the UV light source to become illuminated for a predetermined second duration while the enclosure is maintained in the closed position. The predetermined second duration may be obtained from a look-up table and may be based on configuration information associated with the device or access sites contained within the device. In this way, following illumination of the ultraviolet light source for the first duration, the ultraviolet light source may be illuminated for the predetermined second duration at predetermined time intervals while the enclosure is maintained in the closed position in order to maintain disinfection of the access sites. In some examples, the second duration may be shorter than the first duration since less UV illumination may be needed to perform the disinfection maintenance phase of the access sites.

At 1146, method 1100 may include locking the device. For example, the controller 120 may send a signal to locking mechanism 126 to cause the locking mechanism to engage with the sealing member 181 so as to lock the enclosure in the closed position while the ultraviolet light source is illuminated. At 1148, method 1100 may include providing an indication that the UV light is on. For example, controller 120 may send a signal to the notification system 124 to actuate one or more indicators in the notification system to provide an indication that the ultraviolet light is illuminated. At 1150, method 1100 may include providing an indication that the access sites within the device are in a non-disinfected state. For example, controller 120 may send a signal to the notification system 124 to actuate one or more indicators in the notification system to provide an indication that the second duration has not yet elapsed and thus the access sites may not yet be in a sufficiently disinfected state.

At 1152, method 1100 includes determining if the second duration has elapsed. For example, the controller 120 may include a timing circuit which determines when/if the second duration of UV illumination has elapsed. If the second duration has elapsed at 1152, then method 1100 proceeds back to 1130 to discontinue the UV light illumination, indicate that the UV light is off, indicate that the access sites are disinfected, and unlock the device so that the disinfected access ports may be accessed by a user.

However, if the second duration has not elapsed at 1152, then method 1100 proceeds to 1154. At 1154, method 1100 includes determining if a transition from the closed position to the open position is detected. If a transition from the closed position to the open position is detected at 1154, then method 1100 proceeds back to 1144 to continue illumination of the UV source to maintain disinfection of the access sites. However, if a transition from the closed position to the open position is detected at 1154 before the second duration has elapsed, then method 1100 proceeds to 1120 described above.

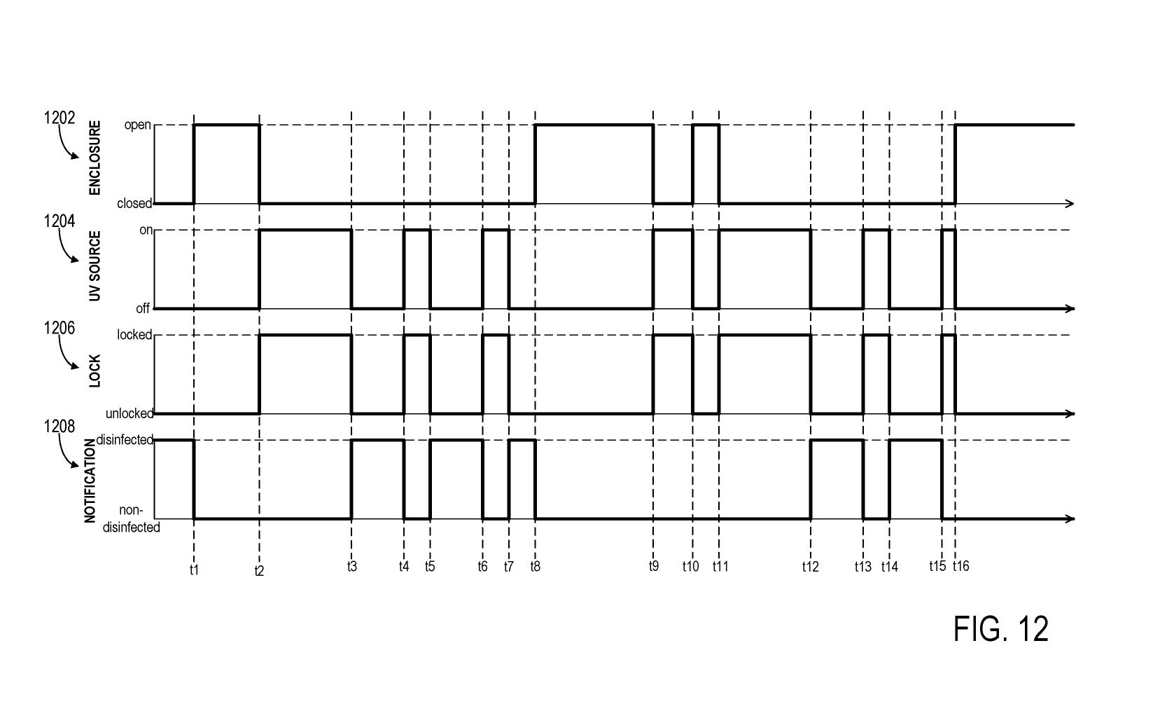

FIG. 12 shows example graphs illustrating a method for maintaining disinfection of access sites of medical devices, e.g., method 1100 described above. In particular, graph 1202 shows adjustments of enclosure 107 between the open position and the closed position versus time. Graph 1204 shows actuation of UV light source 118 versus time. Graph 1206 shows actuation of locking mechanism 126 versus time. Graph 1208 shows actuation of notification system 124 versus time.

Before time t1 in FIG. 12, the enclosure is in the closed position, the UV source is off (not illuminated), the device is unlocked, and the notification system indicates a disinfected state of the access ports. At time t1, the enclosure is opened, e.g., a user may open the enclosure in order to access ports of a catheter in order to administer fluids to a patient or perform other procedures on access sites included in the enclosure. Since the enclosure is opened at time t1 the UV light source is maintained off, the device is maintained unlocked, and the notification system is updated to indicate that the access ports are in a non-disinfected state.

At time t2, the enclosure is closed around the access ports, e.g., following use of the access ports by a user. In order to disinfect the access ports in the enclosure, the UV light source is illuminated for a predetermined first duration from time t2 to t3 in order to adequately disinfect the access sites within the enclosure. During this time period from time t2 to t3, the device is maintained locked in order to prevent a user from opening the device while disinfection is being performed and the notification system provides an indication that the access sites are in a non-disinfected state.

At time t3, the first time duration has elapsed and the access sites are sufficiently disinfected, thus the device is unlocked and the notification system provides an indication that the access sites are sufficiently disinfected for use. After a predetermined time interval from time t3 to time t4, the UV light is again illuminated but for a shorter second time duration from t4 to t5 in order to maintain disinfection of the access sites in the enclosure. During this second duration from t4 to t5, the device is locked and the notification system indicates that the access ports are in a non-disinfected state. After the maintenance disinfection, at time t5 illumination of the UV source is discontinued, the device is unlocked, and the notification system provides an indication that the access ports are disinfected.

In order to continue maintaining disinfection of the access ports, the UV light is cycled on and off periodically. For example, following a time duration after t5, the UV light source is again illuminated for the second duration from time t6 to t7 during which time the device is locked and the notification system indicates that the access sites are non-sterilized or not sufficiently disinfected. After completion of the maintenance disinfection, at time t7 the illumination of the UV source is discontinued, the device is unlocked, and the notification system provides an indication that the access sites are sufficiently disinfected or sterilized.

At time t8, the enclosure is again opened. Since the enclosure is opened at time t8, the UV light source is maintained off, the device is unlocked, and the notification system is updated to indicate that the access sites are in a non-disinfected state. At time t9 the enclosure is closed around the access ports and the UV light source is illuminated to disinfect the access sites within the enclosure. However, at time t10 the device is opened prematurely, before a sufficient time duration of UV illumination has elapsed to sufficiently disinfect the access sites. For example, a user may press an override button to access the access sites in an emergency before disinfection is complete. Thus, at time t10 the UV illumination is terminated, the device is unlocked, and the notification system provides a warning that the access ports are in a non-disinfected state. The enclosure is then closed again at time t11 at which time the UV light source is illuminated for the predetermined first duration from time t11 to t12 in order to adequately disinfect of disinfect the access sites within the enclosure. During this time period, the device is maintained locked and the notification system provides an indication that the access ports are in a non-disinfected state.

At time t12, the first time duration has elapsed and the access sites are sufficiently disinfected, thus the device is unlocked and the notification system provides an indication that the access sites are sufficiently disinfected for use. After a predetermined time interval from time t12 to time t13, the UV light is again illuminated but for the shorter second time duration from t13 to t14 in order to maintain disinfection of the access sites in the enclosure. During this second duration from t13 to t14, the device is locked and the notification system indicates that the access ports are in a non-disinfected state. After the maintenance disinfection, at time t14 the illumination of the UV source is discontinued, the device is unlocked, and the notification system provides an indication that the access ports are sufficiently disinfected or sterilized.

In order to continue maintaining disinfection while the device remains closed around the access ports, following a time duration after t14 the UV light source is again illuminated at time t15. However, at time t16 the device is again opened prematurely before a sufficient time duration of UV illumination has elapsed to sufficiently maintain disinfection of the access ports. For example, a user may press an override button to access the access sites in an emergency before disinfection maintenance is complete. Thus, at time t16 the UV illumination is terminated, the device is unlocked, and the notification system provides a warning that the access ports are in a non-disinfected state.

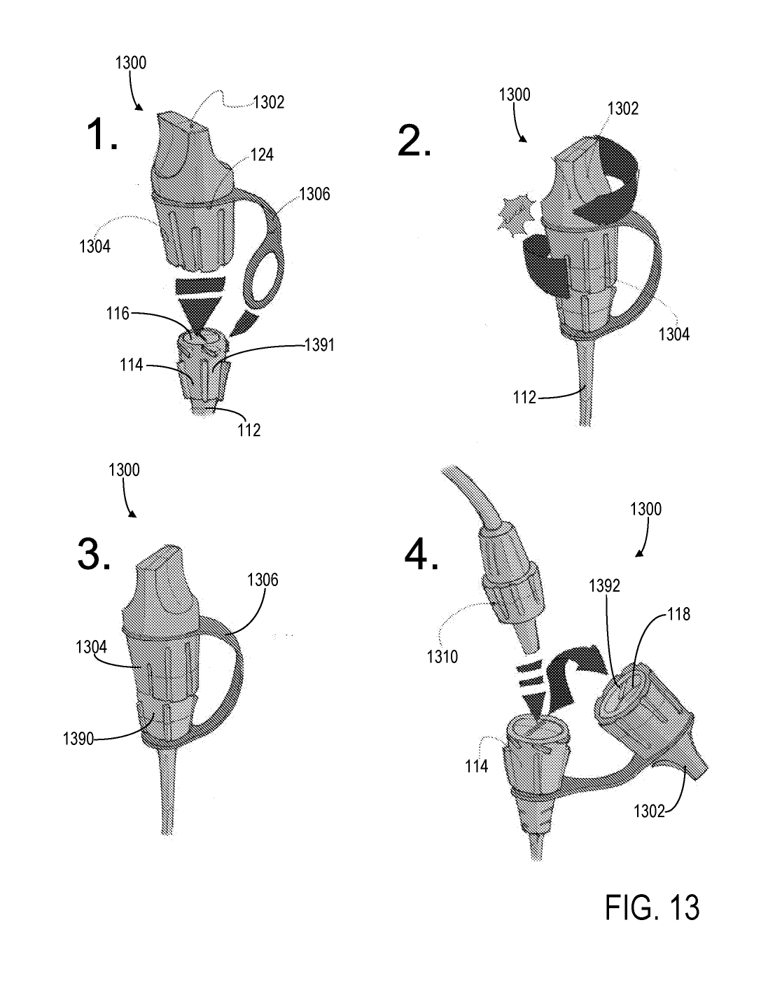

FIGS. 13-26 show various additional example embodiments of a device for maintaining disinfection of access ports of medical devices in accordance with this disclosure. Like-numbered elements appearing in FIGS. 13-26 correspond to like-numbered elements shown in FIGS. 1-9 described above. It is to be understood that these embodiments are exemplary in nature and are not to be considered in a limiting sense, because numerous variations are possible. Further, it should be understood that various features/components or combinations of features/components in the various embodiments described herein may be combined and/or omitted in other embodiments without departing from the scope of this disclosure. For example, various features/components of a first embodiment may be combined with various features/components of a second embodiment without departing from the scope of the disclosure.

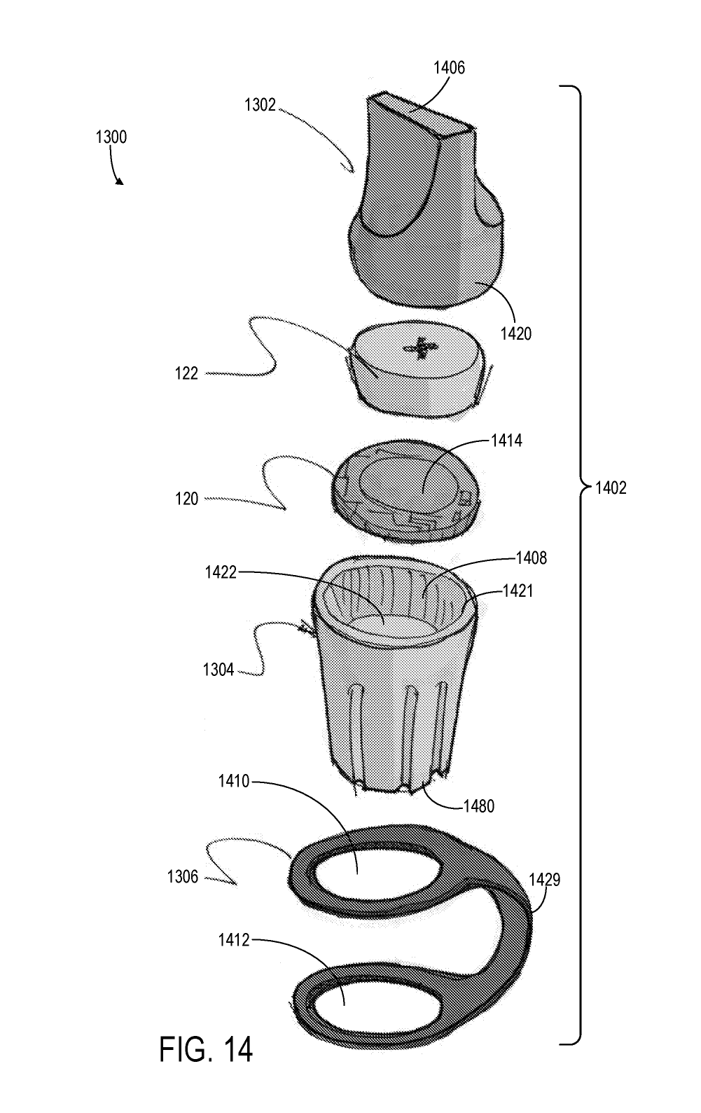

FIGS. 13 and 14 show various viewpoints and operational states of an example embodiment 1300 of device 100 that takes the form of a tethered cap that may be attached to an end 114 of a medical device 112, e.g., a central line port, in order to disinfect an access site 116 of the device. In particular, FIG. 13, illustrates an attachment of embodiment 1300 to a port of a medical device (1), actuation of the device (2), UV disinfection of an access site 116 via the device (3), and removal of the device following sterilization of the access site to connect an incoming medical device, e.g., an incoming intravenous (IV) line. FIG. 14 shows an exploded view 1402 of the various components included in embodiment 1300.

Embodiment 1300 comprises a top cap 1302 coupled to a top portion or top surface of a bottom housing 1304. Top cap 1302 may include features that assist a user in grasping and turning the top cap. For example, top cap 1302 may be shaped like a “gas cap” (e.g., as used to seal a gas tank in an automobile) wherein two opposing sides of the cap taper inwardly in a direction towards the top of the cap to form two opposing indentations in the opposing sides of the cap. The tapered sides of the top cap may terminate at a handle element 1406 having a flat rectangular top surface. However, it should be understood that top cap 1302 may take any suitable shape. For example, top cap 1302 may be cylindrical or box-shaped and may include various other features that assist a user in grasping and rotating the cap.

A bottom portion 1420 of top cap 1302 includes features that are configured to mate with a top portion 1421 of bottom housing 1304. For example, a bottom portion of cap 1302 may include exterior threads configured to mate with interior threads on inner walls within an aperture 1408 in a top portion of bottom housing 1304 so that cap 1302 may be screwed or twisted into the aperture 1408 in housing 1304. As another example, cap 1302 may be coupled with a top portion of housing 1304 via a ratchet mechanism that allows a rotary motion of the cap relative to the housing in only one direction while substantially preventing rotary motion of the cap relative to the housing in the opposite direction. For example, a bottom portion of cap 1302 may include a gear mechanism configured to interface with pawl elements or vertical grooves in the interior walls of a top portion of housing 1304 in aperture 1408.

Aperture 1408 may be sized to hold and/or support various components of the device when the cap 1302 is coupled to the top portion of housing 1304. For example, as shown in FIG. 14, a circular printed circuit board (PCB) 120 may be installed within aperture 1408. In this example a diameter of the circular PCB 120 may be approximately the same as or slightly less than a diameter of aperture 1408. A bottom surface of PCB 120 may be supported by an internal surface 1422 of the aperture 1408 within housing 1304. Additionally, one or more cylindrical batteries, e.g., battery 122, may be installed on a top surface of the PCB 120. In some examples, a bottom surface of cap 1302 may also include an aperture sized to accommodate at least of portion of the one or more batteries, e.g., a portion of battery 122, and/or PCB 120. It should be understood that, though element 120 is shown as a PCB in FIG. 14, element 120 may comprise any suitable controller 120 as described above with regard to FIG. 1. In this example, the top surface of PCB 120 includes a central circular conductor 1414 having a diameter less than the diameter of the PCB. The circular conductor 1414 physically touches a bottom terminal of battery 122 when the cap is coupled to the housing with the battery and PCB installed within. Additionally, when the cap is coupled to the housing with the battery and PCB installed within, one or more conductors in the interior of the bottom portion of the cap may be configured to place the top terminal of the battery in communication with the PCB when the cap is rotated by a predetermined amount relative to the housing to thereby initiate UV disinfection of an access site to which the device is coupled. F or example, a UV light source 118 may be in electrical communication with PCB 120 and PCB 120 may be configured to implement various disinfection routines, examples of which are described herein.

Embodiment 1300 additionally includes a tether 1306 having two opposing eyelets 1410 and 1412 coupled together by an elongated flexible attachment member 1429. For example, tether 1306 may comprise a cord coupled to two opposing rings or tether 1306 may comprise a piece of material with holes formed in each of the opposing ends. Tether 1306 may have any suitable shape and may be composed of any suitable flexible material including but not limited to rubber, flexible plastic, flexible polymer, leather, etc.

Tether 1306 is configured to couple embodiment 1300 of an injection port sterilizer to an end of an injection port or other medical device having an access port. In particular, eyelet 1410 has an internal diameter sized to accommodate a diameter of the components that couple cap 1302 with the top portion of housing 1304. For example, an outer diameter of eyelet 1410 may be slightly greater than the outer diameters of the bottom portion 1420 of cap 1302 and the top portion 1421 of housing 1304 and an inner diameter of eyelet 1410 may be slightly less than the outer diameters of the bottom portion 1420 of cap 1302 and the top portion 1421 of housing 1304. As illustrated at 1 in FIG. 13, eyelet 1410 may be sandwiched between a bottom surface of cap 1302 and a top surface of housing 1304 when the cap is coupled with the housing. The other eyelet 1412 may have a diameter sized to receive an end of a line port or other medical device. For example, as illustrated in FIG. 13, eyelet 1412 may be installed beneath a hub 1390 of a central line access port so that the device remains physically attached to the line via tether 1306.

A bottom portion 1480 of housing 1304 opposing cap 1302 may include an aperture 1392 having internal threads or other coupling members configured to mate with corresponding threads or coupling members on an end 114 of medical device 112. For example, as illustrated in FIG. 13, end 114 of line 112 includes external threads 1391 that are configured to mate with interior threads within an aperture in the bottom portion of housing 1304 so that the main body of the sterilization device may be screwed or twisted onto the end 114 of the line 112 as illustrated at 2 in FIG. 13. The aperture 1392 in the bottom portion of housing 1304 defines a chamber including a UV light source 118, e.g., one or more UV lamps, that may be used to disinfect the access site 116 of line 112 when the sterilization device is attached to the end of the line and actuated as shown at 2 in FIG. 13.

In operation, as illustrated at 2 in FIG. 13, a user may attach the sterilization device to an end 114 of line 112 by placing the end 114 of the line into the bottom aperture 1392 of housing 1304 so that the internal threads in the bottom of the housing engage with external threads on the end of the line. The user may then apply a torque to rotate the device relative to the end 114 to thereby screw or twist the main body of the device onto the port. The cap 1302 may additionally function as an actuation device once the main body is coupled to the port. In particular, the user may continue applying a torque to the cap in the same direction as the torque used to screw the body onto the port to actuate an internal switch that initiates a UV disinfection routine. For example, continued rotation of the cap 1302 following attachment of the device to the end of the medical device may cause the UV light source in the device to become illuminated to disinfect the access site. In some example, the device may include an interference member that produces a sound, e.g., a “click” sound, indicating that the cap has been sufficiently rotated to actuate the device. An on/off indicator 124 may be included in a side of housing 1304. For example, on/off indicator may comprise a LED light or other suitable visual indicator. The on/off indicator 124 may be illuminated when the UV light source is turned on and illumination of indicator 124 may be discontinued when the UV light source is turned off, e.g., via counter rotation of cap 1302.

As illustrated at 3 in FIG. 13, the on/off indicator 124 may remain illuminated while the device is in operation to disinfect the access site. Once a sufficient period of time has elapsed, e.g., 1-5 minutes or when the access site is suitably disinfected, illumination of the on/off indicator 124 may be discontinued to indicate that the access port is sufficiently sterilized. As illustrated at 4 in FIG. 13, following the sterilization by the device, the user may detach the device from the end 114 of line by applying a counter torque to cap 1302 to unscrew the device from the end 114 of the line. The user may then attach an incoming line (e.g., an I.V. line), or otherwise use the now sterilized access port.

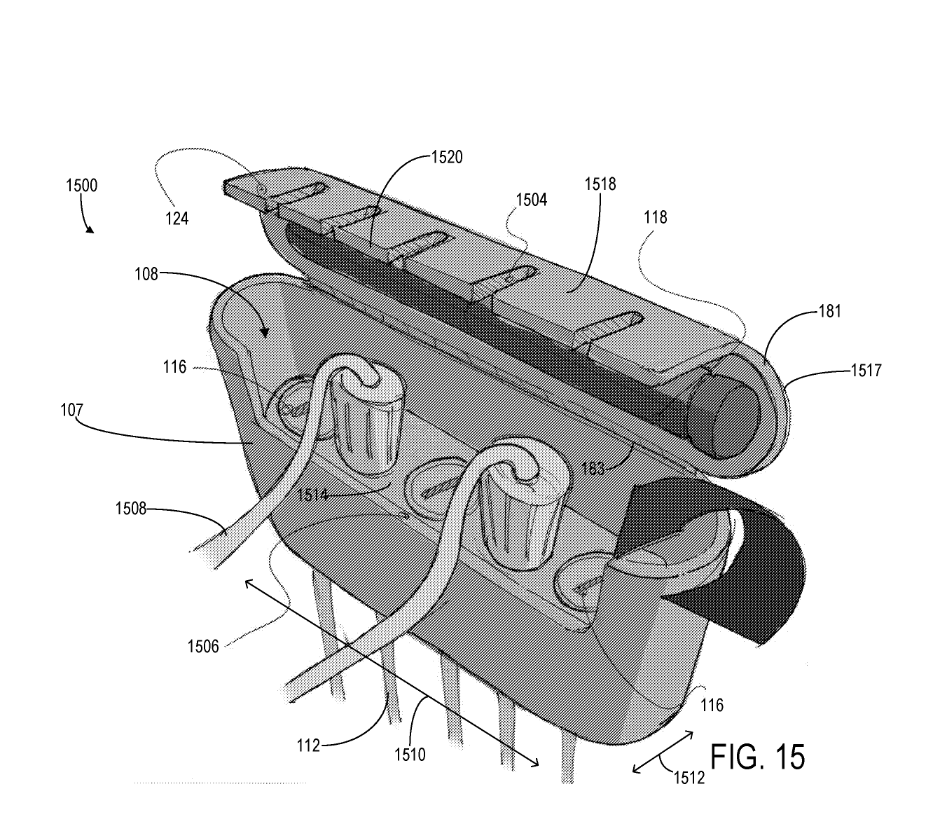

FIG. 15 shows another example embodiment 1500 of a device for maintaining disinfection of access sites of medical devices. Embodiment 1500 comprises an enclosure 107 configured to hold a plurality of access sites (e.g., access site 116) of medical devices, e.g., medical device 112. For example, embodiment 1500 may be configured to hold central line port hubs, needleless injection sites, or other lines having access ports within an interior chamber 108 of the device for disinfection. In this example, enclosure 107 comprises a body having a length 1510 greater than a width 1512. Embodiment 1500 includes a top flap 181 that extends the length of the enclosure and is rotatably coupled, e.g., via a hinge 183, to a back top edge of the enclosure. FIG. 15 shows the top flap 181 in an opened position so that the access sites 116 are exposed.

Ports may be snapped into the enclosure from beneath the enclosure so that the access sites 116 are held in a flat internal bottom surface 1514 within chamber 108. An opening 1506 is formed in the front side of enclosure 107 to provide increased access to the access sites 116 included in chamber 108 when the top flap 181 is in the open position. For example, as shown in FIG. 15, one or more lines 1508 may be coupled to one or more access sites in the chamber when the flap is open. The opening 1506 may be formed as a cut-out in the front side of the enclosure such that the height of the outer walls of the enclosure beneath the opening is less than the height of the outer walls of the enclosure elsewhere.

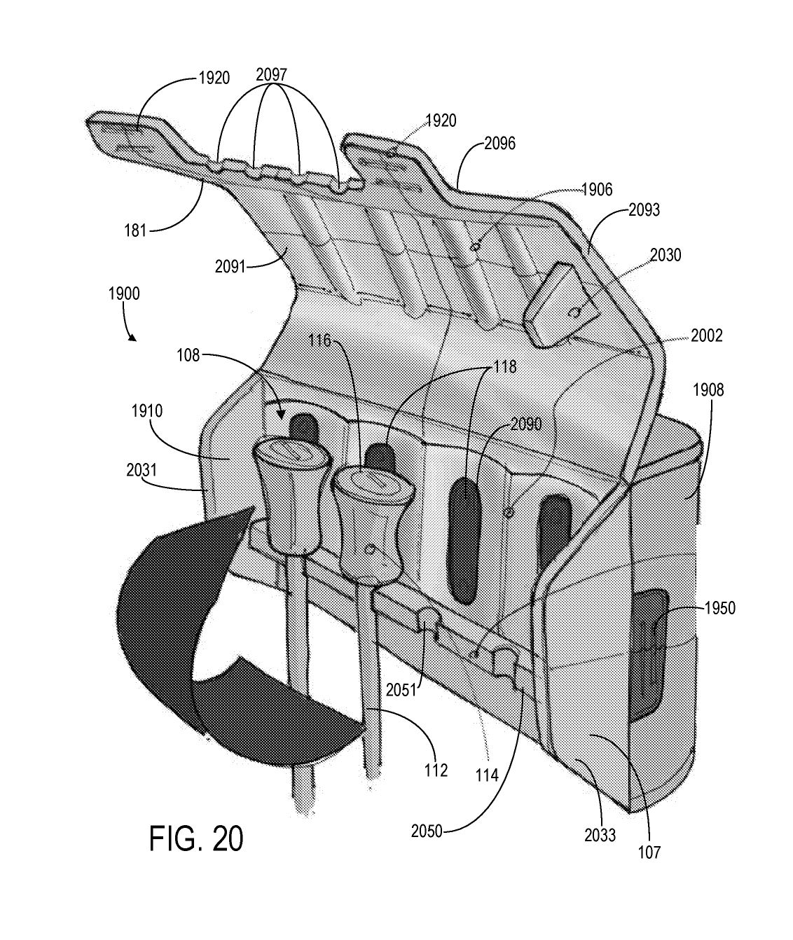

One or more UV light sources 118, e.g., UV fluorescent lamps, may be coupled to a bottom surface of top flap 181. In some embodiments, as shown in FIG. 15, a single UV lamp may be coupled to the bottom surface of the top flap and may extend along the length of the flap above every access site location on the internal bottom surface 1514 of the chamber 108. However, in other examples (e.g., as illustrated in FIG. 20) a plurality of separate UV light sources may be coupled beneath the top flap. In this example, each UV light source in the plurality of separate UV light sources may be positioned on the bottom surface of the top flap at a position that aligns with (e.g., is directly above) a corresponding access site location on the internal bottom surface 1514 of the chamber.

The top flap 181 of embodiment 1500 comprises a top portion 1517 beneath which the UV light source(s) are coupled and a bent portion 1518 that forms an angle with the top portion. For example, the bent portion 1518 may form an angle of approximately 90 degrees relative to the top portion 1517 of the flap such that, when the top flap is moved to a closed position, the bent portion lies in a plane substantially parallel with the plane in which the front surface of the enclosure lies. When in the closed position, the bent portion of the top flap may overlap the opening 1506 in the enclosure.

The bent portion additionally includes slots, notches, or grooves 1504 formed therein. The grooves 1504 comprise openings extend a distance into the bent portion from a bottom edge 1520 of the bent portion. Lengths and widths of the grooves are sized to permit any lines or tubes connected to access ports mounted in the enclosure to exit the enclosure while the top flap is in the closed position. In some examples, the lateral positions of the grooves along the length of the bent portion of the top flap may substantially align with the access site locations on the internal bottom surface 1514 of the chamber such that there is a groove for each access site location in the enclosure. For example, if the device is configured to hold four access ports then there may be four grooves included in the bent portion of the top flap. However, any suitable number and positioning of grooves in the top flap may be used, e.g., there may be a greater or lesser number of grooves than access site locations.

Top flap 181 additionally includes a visual indicator 124, e.g., an LED light or other light source. In some examples, visual indicator 124 may be illuminated when the top flap is closed and when the UV light source(s) is illuminated. Illumination of the visual indicator 124 may be discontinued once the access ports are suitably disinfected while the top flap is in the closed position.

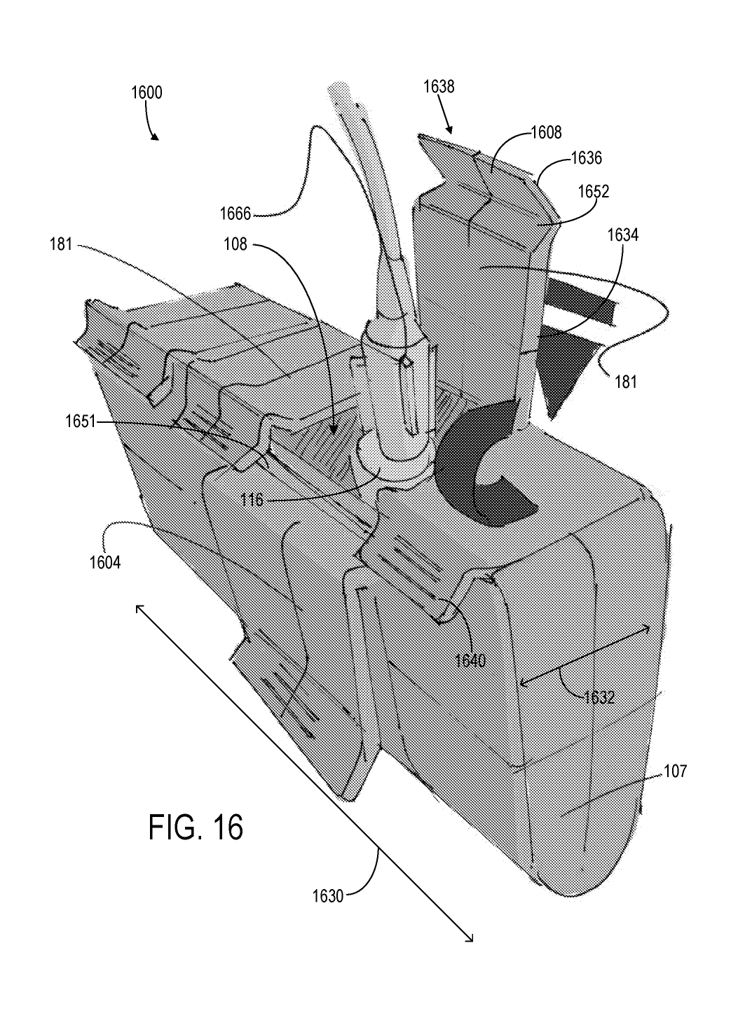

FIG. 16 shows another example embodiment 1600 of a device for maintaining disinfection of access sites of medical devices that takes the form of an individual port source array. As in embodiment 1500 shown in FIG. 15 described above, embodiment 1600 comprises an enclosure 107 configured to hold a plurality of access sites (e.g., access site 116) of various medical devices. Enclosure 107 is formed as a body having a length 1630 greater than a width 1632. In this example, a plurality of top flaps or hatches 181 are rotatably coupled to a back top edge of the enclosure above each access site location in the enclosure. Each flap may include a flat top portion 1634 and a bent portion at the front end 1638 of the flap. The bent portion may be configured to snap over a front top edge 1651 of the enclosure to hold the flap in a closed position. For example, when closed by a user, an angle 1652 formed in the bent portion may temporarily increase when a force is applied to press the flap down onto the front top edge of the enclosure to snap the bent edge around the front top edge. From the closed position, a user may open the flap by pulling upward on a front tab 1640 of the flap. When the flap is fully opened, the flap may be slid down into an aperture included in a back portion of the enclosure behind the flap.

The chamber 108 formed by enclosure 107 may include one or more UV light sources for disinfecting access sites. In some examples, each access site location in enclosure 107 may have its own UV light source. In this example, when a flap positioned above an access site location in the enclosure is moved to the closed position, the device may be configured to illuminate the UV light source associated with that access site location and discontinue illumination of the UV light source when the flap is opened or when a sufficient period of time has elapsed to sufficiently disinfect an access site at the access site location. After disinfection of an access site, the corresponding flap may be opened so that the access site may be used, e.g., to connect a line 1666 to the access site.

Though not shown in FIG. 16, the embodiment 1600 may include various notification systems or components, e.g., LED lights, timers, etc. that indicate various operational states of the device. Additionally, in this example, a clip 1604 is included on a front surface of enclosure 107. For example, clip 1604 may be used to attach the device to clothing, bedding, etc., while the device is in use.

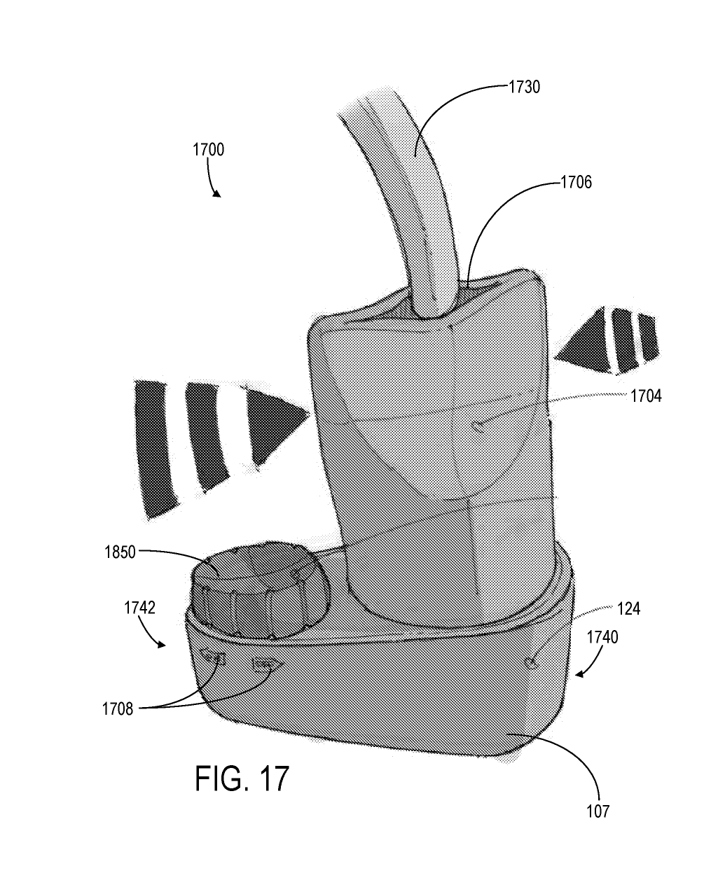

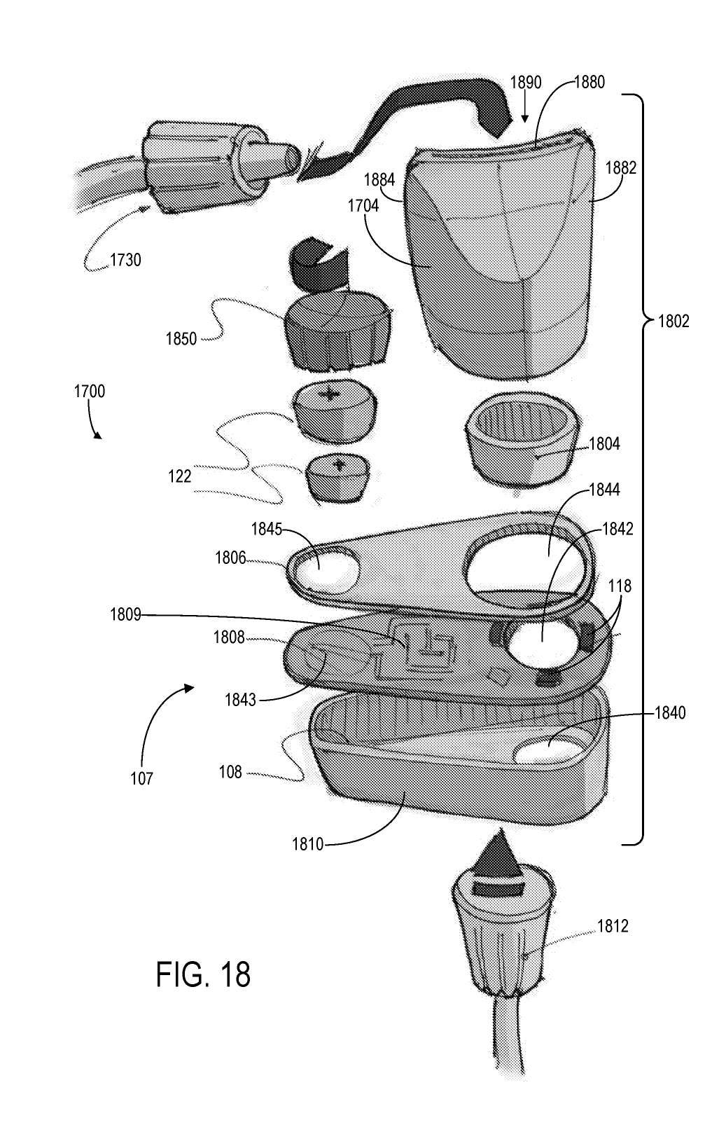

FIGS. 17 and 18 show yet another example embodiment 1700 of device 100 that takes the form of a small device having a light protector 1704 that can be attached to an end of a medical device, e.g., an inlet port 1812, to maintain disinfection of an access site of the medical device. In particular, FIG. 17 shows a perspective view of embodiment 1700 and FIG. 18 shows an exploded view 1802 of various components of embodiment 1700.

Embodiment 1700 comprises a coffin-shaped enclosure 107 have a first rounded end 1740 and a second smaller opposing rounded end 1742. As shown in FIG. 18, the enclosure 107 comprises a base housing 1810 defining a chamber 108 and having a circular aperture 1840 in a bottom surface adjacent to end 1740. A diameter of aperture 1840 is sized to receive an end of medical device 1812, e.g., an inlet port on the patient side. The end of device 1812 may be coupled to aperture 1840 in any suitable manner. For example, the end of medical device 1812 may have external threads configured to mate with threads in the sterilization device.

A coffin-shaped component 1808 comprising circuitry 1809 configured to implement the various disinfection routines described herein may be installed within base housing 1810. A circular aperture 1842 adjacent to end 1740 is formed in component 1808. A diameter of aperture 1842 may be sized to receive at least a portion of an end of medical device 1812. One or more UV light sources, e.g., LED UV lights, may be mounted on component 1808 adjacent to and around the internal edges of aperture 1842. In some examples, a plurality of UV light sources may be equally spaced around the circumference of aperture 1842. Any suitable number of UV light sources may be included on component 1808 in any suitable manner, e.g., 2, 4, or 6 equally spaced UV light sources may be mounted to component 1808 around aperture 1842. Component 1808 also includes a circular conductor 1842 mounted to a top surface of component 1808 adjacent to end 1742.

Enclosure 107 also includes a coffin-shaped top cap 1806 that is installed on top of component 1808 and coupled to the top edges of base housing 1810. Top cap 1806 includes a circular aperture 1844 at end 1740 that has a diameter larger than apertures 1842 and 1840. The diameter of aperture 1844 is sized to receive and hold a lens 1804. Lens 1804 may direct light from UV light sources 118 toward a flexible boot 1704 mounted on a top surface of top cap 1806. A smaller aperture 1845 is included in top cap 1806 adjacent to end 1742. Aperture 1845 has a diameter smaller than aperture 1844 and is sized to permit a terminal of a cylindrical battery (e.g., batteries 122) to be in physical contact with conductor 1843 in component 1808.

A battery cap 1850 is coupled to top cap 1806 above aperture 1845. Battery cap 1850 comprises a substantially hollow cylindrical body having a diameter sized to contain at least a portion of batteries 122 when the battery cap is rotatably coupled to top cap 1806. A conductor may be included in battery cap 1850 so that the top cap functions as a switch to actuate the device. For example, when cap 1850 is in a first position, the conductor may not provide an electrical connection between the top terminal of the top battery and component 1808, and when cap 1850 is rotated to a second position the conductor may provide an electrical connection between the top terminal of the top battery and component 1808. In this way, battery cap 1850 may function as an actuating mechanism (an on/off switch) such that when a user rotates the battery cap from a first position to a second position, the UV light source is illuminated to disinfect any access ports coupled within the device. Conversely, when a user applies a counter rotation to the battery cap, UV light illumination may be discontinued.

The flexible boot 1704 is coupled to top cap 1806 a locations adjacent the edges of aperture 1844 and over lens 1804. A bottom portion of boot 1704 may be coupled to top cap in any suitable manner, e.g., via an adhesive, an interference fit, mechanical coupling components, etc. Boot 1704 comprises a flexible material having a slit 1880 at a distal end 1890 of the boot opposing the top cap. The boot may be composed of any suitable flexible material, e.g., rubber, flexible plastic, leather, flexible polymer, ethylene vinyl acetate (EVA), etc. Boot 1704 may have front and back faces that taper inwardly towards each other to terminate at the elongated slit 1880. The slit 1880 may be substantially perpendicular to the tapering faces and the opposing ends of the elongated slit may terminate at two opposing sides 1884 and 1882 of the boot, where the two opposing sides 1884 and 1882 are substantially perpendicular to the tapered front and back faces of the boot. The slit 1880 is biased to remain in a closed position. A user may open slit 1880 by applying inward forces to the two opposing sides 1884 and 1882 of the boot. For example, a user may press opposing sides 1882 and 1884 towards each other to temporarily open the slit to insert an outlet port 1730 into the boot to be connected to an inlet port 1812 coupled to the device.

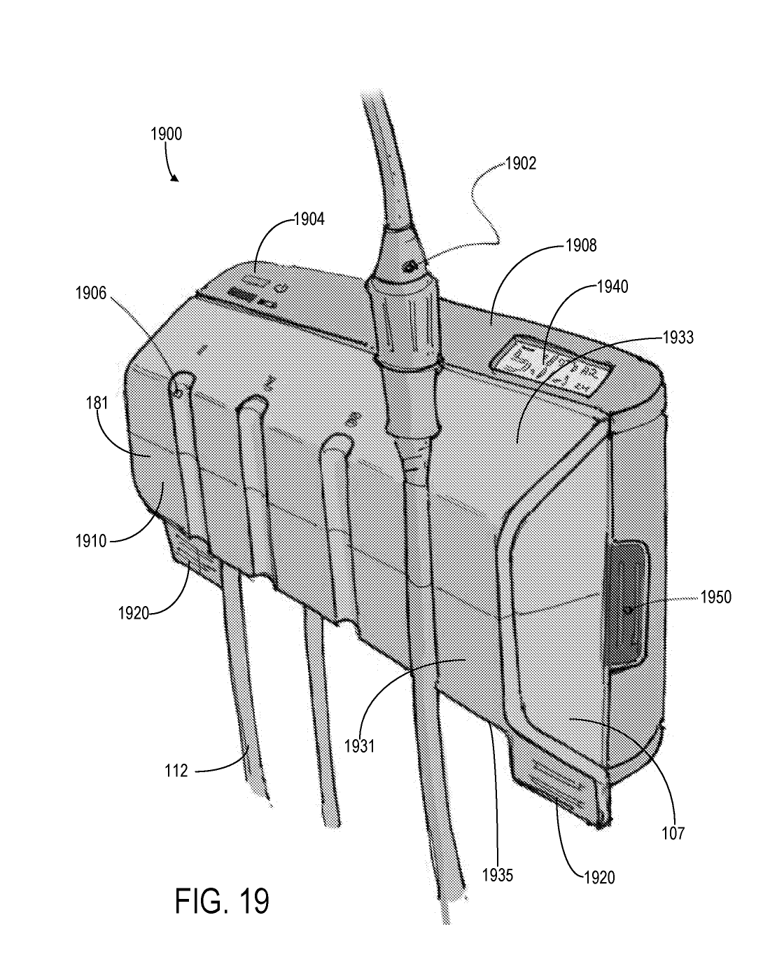

FIGS. 19-22 show various operational states of another example embodiment 1900 of a device for sterilizing access sites of medical devices. Embodiment 1900 comprises a receiving module 1910 (e.g., a consumable component) that is releasably coupled to a transmitting module 1908 (e.g., a source component). Receiving module 1910 corresponds to receiving module 102 described above with regard to FIG. 1 and transmitting module 1908 corresponds to transmitting module 104 described above with regard to FIG. 1.

Receiving module 1910 comprises an enclosure 107 configured to hold a plurality of access sites (e.g., access site 116) of various medical devices (e.g., line 112). Enclosure 107 comprises two opposing side walls 2031 and 2033 and a flap 181 that is adjustable between an open position (as shown in FIG. 20) and a closed position (as shown in FIG. 19). Flap 181 is rotatably coupled to a back top edge of enclosure 107. Flap 181 has a front flat surface 1931 extending into obliquely angled top and bottom surfaces, 1933 and 1935 respectively. For example, the top and bottom surfaces 1933 and 1935 may both form an angle greater than 90 degrees relative to the front face 1931 of flap 181. In the closed position, the outer lateral sides of the flap overlap the edges of the two opposing side walls 2031 and 2033.

Enclosure 107 forms a chamber 108 within which ends of medical devices may be mounted for disinfection. In particular, enclosure 107 includes an internal platform 2050 adjacent to a bottom side of the enclosure and extending from a back wall of the enclosure towards a front side of the enclosure. Platform 2050 may include one or more notches or snap slots, e.g., slot 2051, sized to hold lines, tubes, or other components for attachment within chamber 108. For example, unused ports may be mounted within chamber 108 via the slots in platform 2050. Each slot in platform 2050 is positioned at a location along the platform to hold an access site directly in front of a corresponding elongated oval aperture in a back wall of the enclosure. For example, slot 2051 is configured to hold an access site directly in front of elongated oval aperture 2090.

A back wall of the enclosure 107 includes one or more elongated oval apertures positioned directly behind each access site location in the enclosure so that when the receiving module 1910 is coupled to the transmitting module 1908, UV light from UV light sources 118 included in the transmitting module 1908 is directed through the oval apertures towards the access site locations in chamber 108. The internal walls of the enclosure are concave around the perimeter of each elongated oval opening in the internal back surface of the enclosure to direct and spread UV light onto the associated access site locations. Additionally, the interior walls of the enclosure may be coated with a reflective material (e.g., chrome or the like) to increase UV light exposure to any access sites mounted in the enclosure.

An interlock tab 2030 is included at a location on an interior surface 2091 of flap 181 adjacent to a side edge 2093 of the flap. Tab 2030 is sized and shaped to fit through an aperture in the back wall of enclosure 107 and into a interlock port 2131 in a front wall of transmitting module 1908 such that when the flap 181 is closed while the receiving module is coupled to the transmitting module, the tab fits into port 2131 to activate the various disinfection routines described herein.

The front face 1931 of flap 181 may include a plurality of vertical slots 1906 (e.g., snap slots) extending substantially the entire height of the front face 1931. Vertical slots 1906 may be sized to receive and hold lines or tubes for mounting therein. For example, when a port 1902 is in use, the tube of the port may be mounted in one of the vertical slots on the front face of the enclosure as shown in FIG. 19. In some examples, indicia may be included on the outer surface of the enclosure adjacent to the each vertical slot 1906, e.g., each slot may be numbered to assist in identification. However, in other examples, indicia may be omitted.

Flap 181 additionally includes tabs 1920 extending from a bottom edge 2096 of the flap to assist a user in opening the flap. Additionally, the bottom edge 2096 of the flap may include slots 2097 for stabilizing and positioning any tubes emanating from chamber 108 when flap 181 is in the closed position. In some examples, these slots 2097 may be positioned along the bottom edge 2096 between two tabs (e.g., tabs 1920) located adjacent to outer side edges of the flap.

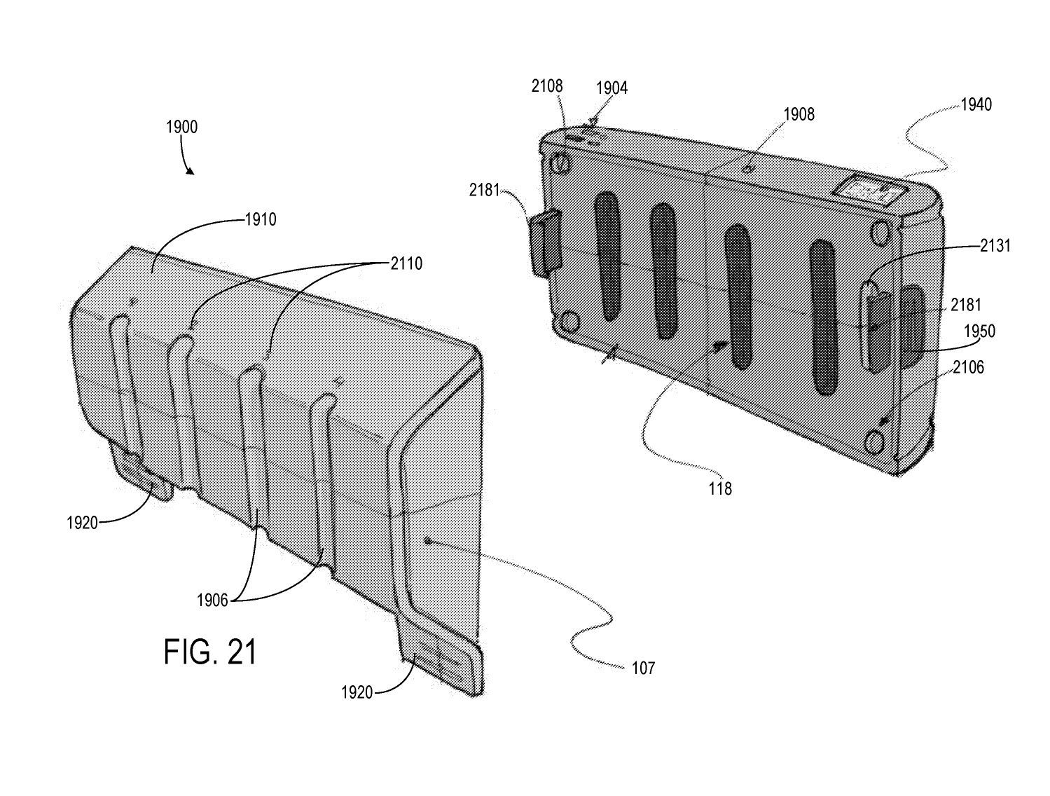

In some examples, receiving module 1910 may be periodically replaced, and thus may be composed of relatively inexpensive materials, e.g., plastics and/or other consumable materials. For example, the receiving module 1910 may be removed from the transmitting module 1908 after a certain number of uses and replaced with a new receiving module. FIG. 21 shows the receiving module 1910 separated from the transmitting module 1908. Receiving module 1910 may be removed from the transmitting module 1908 via user actuation of a release button 1950 included on a side of the transmitting module. Release button 1950 may be in mechanical communication with two opposing release lock tabs 2181 positioned adjacent to two opposing sides of the transmitting module 1908. Release lock tabs 2181 may be configured to releasably engage with corresponding slots included in a back wall of receiving module 1910. For example, when a user presses the release button 1950, the release lock tabs 2181 may disengage the receiving module so that the two components can be separated as shown in FIG. 21.

Transmitting module 1908 may include sockets 2108 positioned adjacent to each corner of a front side of the transmitting module 1908. Sockets 2108 may be sized and shaped to receive corresponding mating components located on the back wall of the receiving module. In some examples, at least one socket may include a sensor configured to detect whether or not a receiving module is coupled to the transmitting module in order to prevent activation of UV light when there is no receiving module attached to the transmitting module.

The transmitting module includes a plurality of elongated oval-shaped UV light sources 118 that are positioned and embedded in a front face of the transmitting module at positions corresponding to the elongated oval apertures (e.g., aperture 2090) in the back wall of enclosure 107. When the receiving module is attached to the transmitting module, the UV light sources align with the apertures in the receiving module so that UV light can be directed onto each individual access site location within enclosure 107.

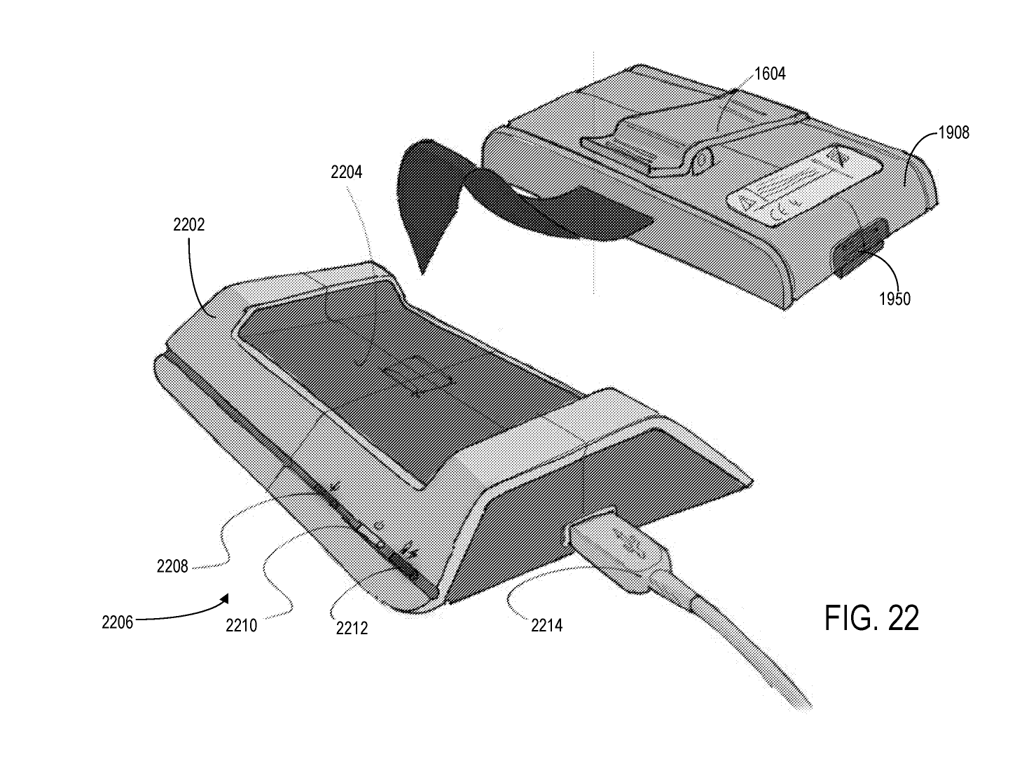

Transmitting module 1908 may include various notification systems or components, e.g., LED lights, timers, displays, etc. that indicate various operational states of the device. For example, one or more LEDs 1904 and/or a display 1940 may be included in a top surface of transmitting module 1908. For example, LEDs 1904 may be illuminated when the device is in operation (e.g., when the UV lights are illuminated) and/or display 1940 may display a timer indicating time information associated with operation of the device, e.g., how much time has elapsed following an initiation of UV light source illumination. Additionally, a clip 1604 may be included on the back surface of transmitting module 1908 as shown in FIG. 22. For example, clip 1604 may be used to attach the device to clothing, bedding, etc., while the device is in use.

After the transmitting module 1908 is separated from the receiving module 1910, in some examples, the transmitting module 1908 may be releasably attached to a docking module 2202 as shown in FIG. 22. Docking module 2202 comprises a platform sized to receive a front face of the transmitting module. When transmitting module 1908 is coupled to the docking module 2202, the docking module may perform a variety of operations on the transmitting module 1908. For example, docking module 2202 may include an induction charge pad to recharge batteries in the transmitting module. As another example, docking module may be used to send and/or receive data from the transmitting module. Docking module may additionally include various ports, e.g., power source ports, USB ports for a USB connection 2214, and/or various notification systems 2206, e.g., a USB data LED 2208, a power LED 2210, and charge LED 2212, to indicate various operational states of docking module 2202.

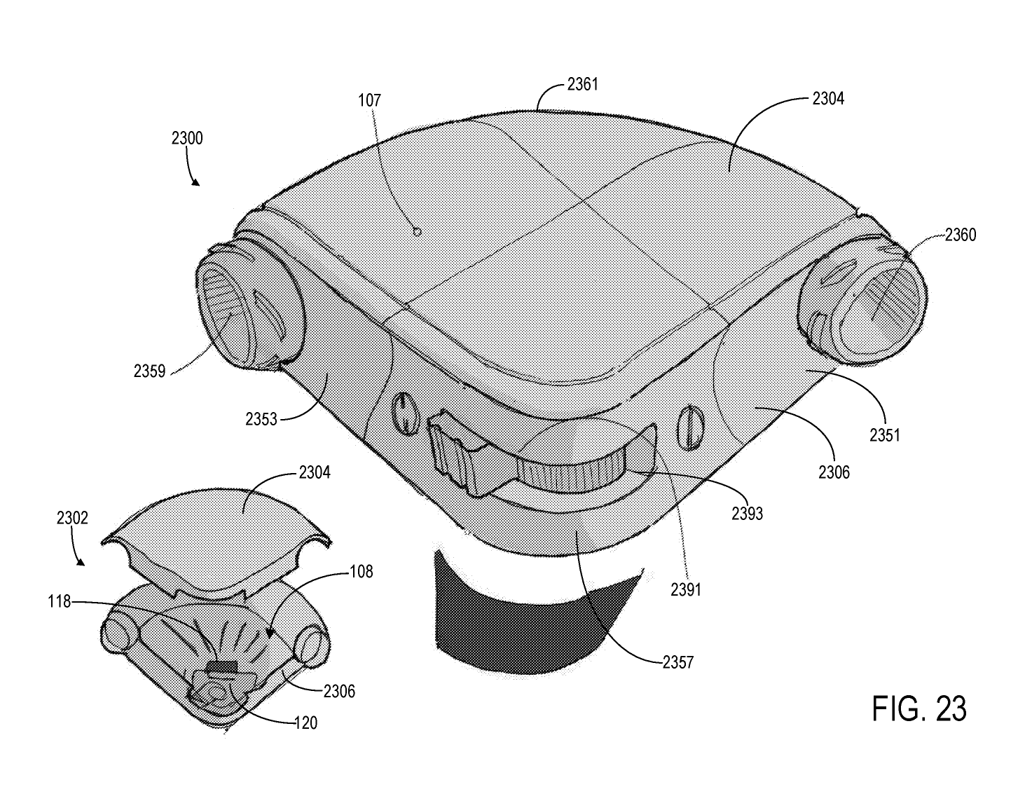

FIG. 23 shows another example embodiment 2300 of a device for sterilizing access ports. Embodiment 2300 comprises a sealed enclosure 107 having a quarter-circle shape. In particular, in this example enclosure 107 comprises a first wall 2351 and a second wall 2353 that intersect at a rounded apex 2357 at the front of the device. The first wall 2351 forms an angle of approximately 90 degrees with the second wall 2352. A back side of enclosure 107 opposite apex 2357 is curved to form a quarter-circle shape when the device is viewed from above. In this example, a first port inlet 2359 is included in wall 2353 adjacent to the back side 2361 of the device and a second port inlet 2360 is included in wall 2351 adjacent to the back side 2361. The first and second port inlets, 2359 and 2360, may comprise cylindrically shaped components that extend outwardly from each side. The first and second port inlets, 2359 and 2360, each include external threads configured to mate with corresponding threads on the ends of medical devices or ports. For example, a first medical port may be coupled to the first port inlet 2359 and a second medical port may be coupled to the second port inlet 2360.

At 2303, FIG. 23 shows an exploded view of embodiment 2300. In this example, enclosure 107 comprises a top plate 2304 having a quarter-circle shape that is attached to a quarter-circle shaped bottom housing 2306 to form a chamber 108. A controller 120 and a UV light source 118 are mounted within the bottom housing 2306 adjacent to the apex 2357 of the device. Additionally a switch 2391 is coupled to controller 120 and extends out of a rectangular aperture 2393 formed in the bottom housing at apex 2357. Switch 2391 may be used to control operation of the device to disinfect any access sites coupled to the first or second port inlets. For example, if a user moves the switch to one side of the device, the UV light source may be illuminated, and if the user moves the switch to the other side of the device illumination of the UV light source may be discontinued.

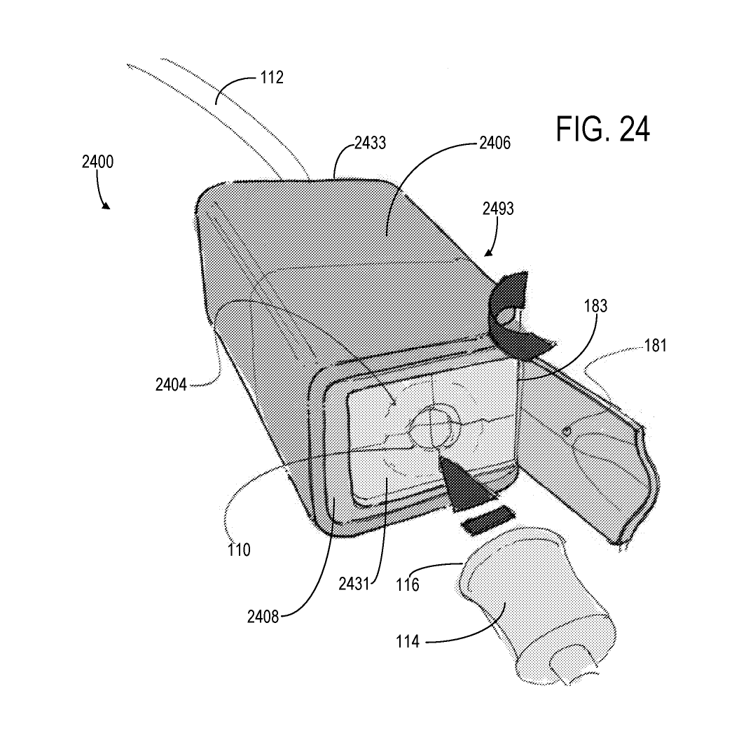

FIGS. 24-26 show various operational states of yet another example embodiment 2300 of a device for sterilizing access ports. In this example, the device takes the form of a stackable double connector having a front port inlet 110 on a square-shaped front face 2431 and a back port inlet on a square shaped back face 2433 to which access ports of medical devices may be coupled. For example, the port inlets may comprise threaded apertures to which access ports may be coupled.

Embodiment 2400 comprises an outer shell 2406 within which a transmitting module 2408 may be housed. Outer shell 2406 comprises substantially parallel top and bottom surfaces, 2531 and 2533 respectively, coupled together by a side surface 2351 that is substantially perpendicular to the top and bottom surfaces. The transmitting module 2408 comprises a rectangular shaped body that includes a controller and one or more UV light sources. Batteries 122 may be coupled to a side of the transmitting module and the transmitting module may be sized to fit within a space defined by the walls of the outer shell 2406. Interior surfaces of the outer shell may include slots 2505 shaped to mate with corresponding components on the outer surface of transmitting module to releasably lock the transmitting module within the outer shell 2406.

In some examples, one or more light lenses may be included within an interior of the transmitting module adjacent to each port inlet to direct UV light from the UV light source(s) towards any access site coupled to the port inlet. Additionally, switches may be included within the interior of the transmitting module adjacent to each port inlet to automatically actuate the device in response to attachment of an access site to a port inlet. For example, an interference component may be included in the transmitting module adjacent to internal threads of a port inlet such that when an access port is twisted into the port inlet, the access port engages the interference component to actuate UV disinfection of the access site.

Transmitting module 2408 also includes doors 181 at each of the front and back faces. Each door at each face may be rotatably coupled to an edge of the face, e.g., via a spring-loaded hinge that biases the door into a closed position over the port inlet in the face.

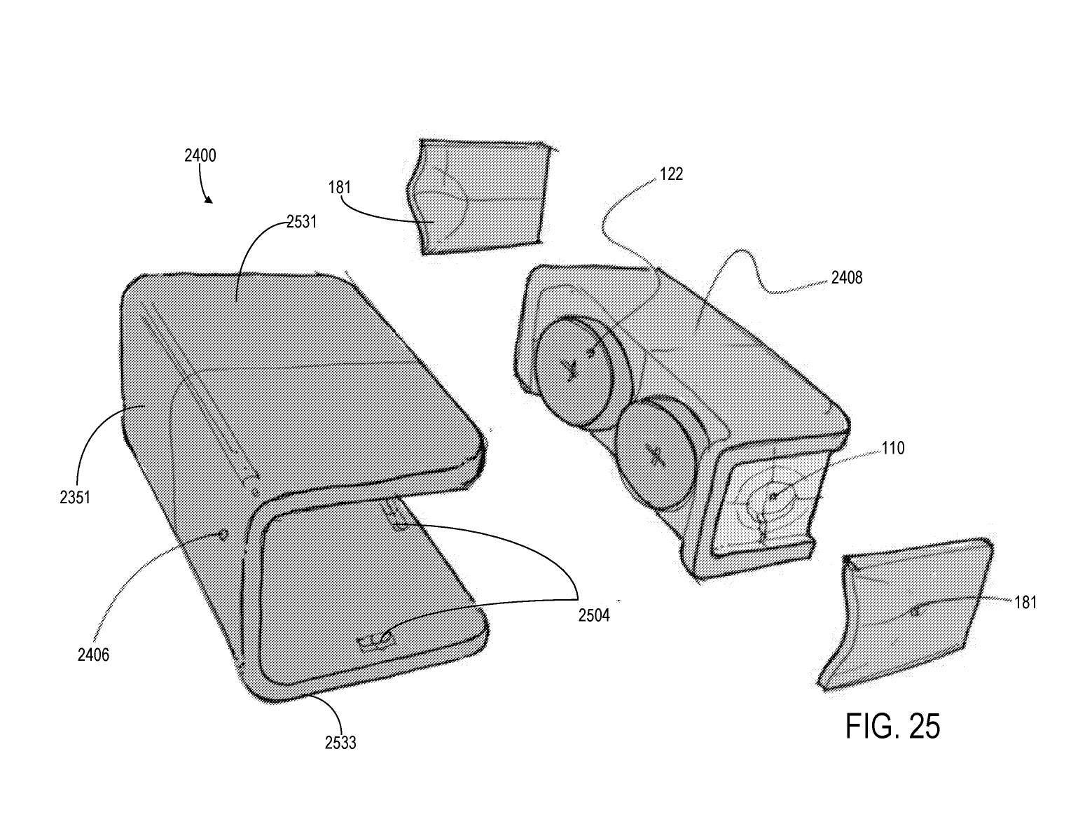

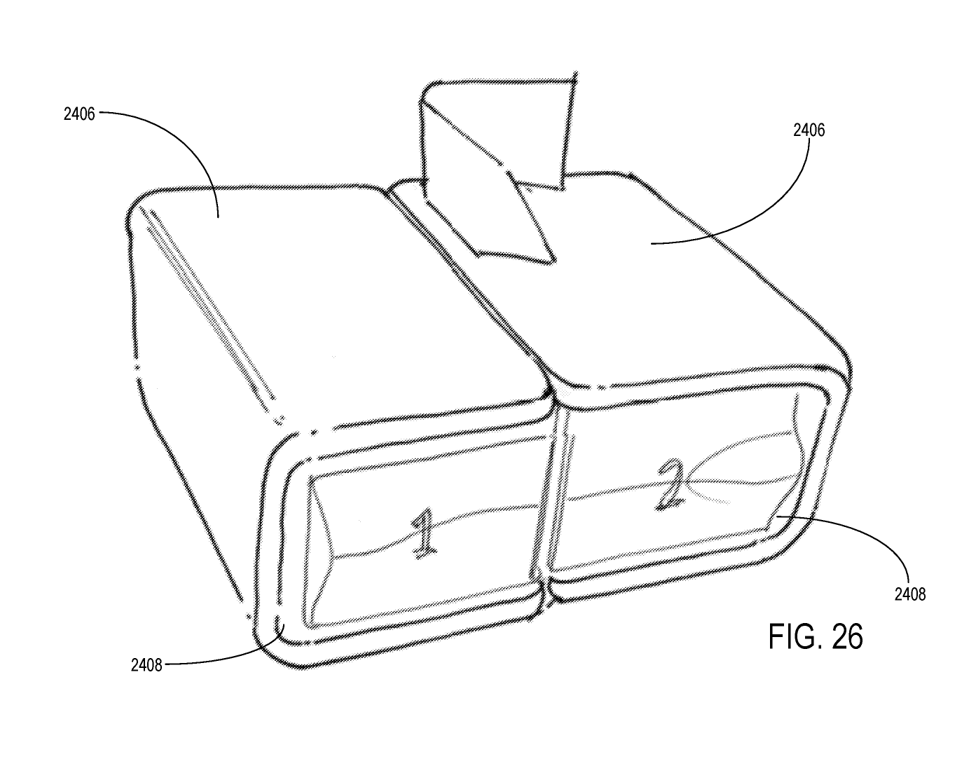

As illustrated in FIG. 26, the embodiments of the device shown in FIGS. 24 and 25 may be stacked together via tracks or other coupling elements included at the open end 2493 of each device. For example, top and bottom edges of the open end 2493 of each device may include tracks configured to couple the two devices together as shown in FIG. 26. For example, a user may slide tracks in the open end of a first device into corresponding tracks in the open end of a second device as illustrated in FIG. 26.

In some embodiments, the above described methods and processes may be tied to a computing system including one or more computers. In particular, the methods and processes described herein may be implemented as a computer application, computer service, computer API, computer library, and/or other computer program product. For example, method 1000 described above may be implemented via controller 120 included in device 100. Controller 120 may be any suitable computing device or microprocessor. As another example, device 100 may be regarded as a computing device configured to maintain disinfection of access ports of medical devices as described above.

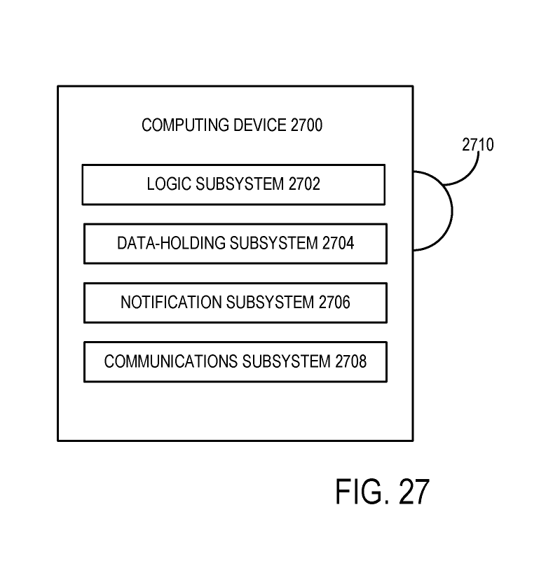

FIG. 27 schematically shows a nonlimiting computing device 2700 that may perform one or more of the above described methods and processes. Computing device 2700 is shown in simplified form. It is to be understood that virtually any computer architecture may be used without departing from the scope of this disclosure. In different embodiments, computing device 2700 may take the form of a printed circuit board (PCB), microcomputer, an integrated computer circuit, microchip, a mainframe computer, server computer, desktop computer, laptop computer, tablet computer, home entertainment computer, network computing device, mobile computing device, mobile communication device, gaming device, etc.

Computing device 2700 includes a logic subsystem 2702 and a data-holding subsystem 2704. Computing device 2700 may optionally include a notification subsystem 2706 and a communication subsystem 2708, and/or other components not shown in FIG. 27. Computing device 2700 may also optionally include user input devices such as manually actuated buttons, switches, keyboards, mice, game controllers, cameras, microphones, and/or touch screens, for example.

Logic subsystem 2702 may include one or more physical devices configured to execute one or more machine-readable instructions. For example, the logic subsystem may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem may include one or more processors that are configured to execute software instructions. Additionally or alternatively, the logic subsystem may include one or more hardware or firmware logic machines configured to execute hardware or firmware instructions. Processors of the logic subsystem may be single core or multicore, and the programs executed thereon may be configured for parallel or distributed processing. The logic subsystem may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration.

Data-holding subsystem 2704 may include one or more physical, non-transitory, devices configured to hold data and/or instructions executable by the logic subsystem to implement the herein described methods and processes. When such methods and processes are implemented, the state of data-holding subsystem 2704 may be transformed (e.g., to hold different data).

Data-holding subsystem 2704 may include removable media and/or built-in devices. Data-holding subsystem 2704 may include optical memory devices (e.g., CD, DVD, HD-DVD, Blu-Ray Disc, etc.), semiconductor memory devices (e.g., RAM, EPROM, EEPROM, etc.), and/or magnetic memory devices (e.g., hard disk drive, floppy disk drive, tape drive, MRAM, etc.), among others. Data-holding subsystem 2704 may include devices with one or more of the following characteristics: volatile, nonvolatile, dynamic, static, read/write, read-only, random access, sequential access, location addressable, file addressable, and content addressable. In some embodiments, logic subsystem 2702 and data-holding subsystem 2704 may be integrated into one or more common devices, such as an application specific integrated circuit or a system on a chip.

FIG. 27 also shows an aspect of the data-holding subsystem in the form of removable computer-readable storage media 2710, which may be used to store and/or transfer data and/or instructions executable to implement the herein described methods and processes. Removable computer-readable storage media 2710 may take the form of CDs, DVDs, HD-DVDs, Blu-Ray Discs, flash drives, EEPROMs, and/or floppy disks, among others.

When included, notification subsystem 2706 may be used to present visual and/or audio and/or haptic representations of data held by data-holding subsystem 2704. As the herein described methods and processes change the data held by the data-holding subsystem, and thus transform the state of the data-holding subsystem, the state of notification subsystem 2706 may likewise be transformed to visually and/or sonically and/or haptically represent changes in the underlying data. Notification subsystem 2706 may include one or more display devices utilizing virtually any type of technology. Such display devices may be combined with logic subsystem 2702 and/or data-holding subsystem 2704 in a shared enclosure, or such display devices may be peripheral display devices. Notification subsystem 2706 may include one or more audio devices, e.g., one or more speakers, and/or one or more haptic devices utilizing virtually any type of technology.

When included, communication subsystem 2708 may be configured to communicatively couple computing device 2700 with one or more other computing devices. Communication subsystem 2708 may include wired and/or wireless communication devices compatible with one or more different communication protocols. As nonlimiting examples, the communication subsystem may be configured for communication via a wireless telephone network, a wireless local area network, a wired local area network, a wireless wide area network, a wired wide area network, etc. In some embodiments, the communication subsystem may allow computing device 2700 to send and/or receive messages to and/or from other devices via a network such as the Internet.

Attachment devices may be included in the system to attach one or more of the above illustrated devices to a patient. For example, an adhesive gel may be provided on an external surface of one of the above housings (e.g., at 2351) for temporary attachment to a patient's skin. The gel may be a hydrogel, and in one example may be a colloid hydrogel patch. Additionally or alternatively, a clip may be provided to engage to a patient's clavicle and or clothing. Further, a shoulder bag may also be coupled with the device so that it may be worn by a patient. Still another example may include a fabric bandage material coupled to the device for coupling to a patient's skin.

It is to be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated may be performed in the sequence illustrated, in other sequences, in parallel, or in some cases omitted. Likewise, the order of the above-described processes may be changed.

The subject matter of the present disclosure includes all novel and nonobvious combinations and subcombinations of the various processes, systems and configurations, and other features, functions, acts, and/or properties disclosed herein, as well as any and all equivalents thereof.

1. A device for maintaining disinfection of access sites of medical devices, comprising:
an enclosure configured to engage an end of a medical device such that an access site of the medical device is positioned within a chamber of the enclosure, the enclosure adjustable between a closed position and an open position, wherein in the closed position the enclosure is configured to enclose the access site within the chamber and wherein in the open position the enclosure is configured to expose the access site;
an ultraviolet light source within the chamber of the enclosure; and
a controller configured to:
in response to an adjustment of the enclosure from the open position to the closed position:
illuminate the ultraviolet light source for a predetermined first duration while the enclosure is maintained in the closed position; and
following illumination of the ultraviolet light source for the first duration, illuminate the ultraviolet light source for a predetermined second duration at predetermined time intervals while the enclosure is maintained in the closed position.

2. The device of claim 1, wherein the controller is further configured to discontinue illumination of the ultraviolet light source in response to an adjustment of the enclosure from the closed position to the open position.

3. The device of claim 1, further comprising a notification system in communication with the controller, wherein the controller is further configured to provide an indication of a non-disinfected state via the notification system when the enclosure is adjusted from the closed position to the open position during the predetermined first duration.

4. The device of claim 3 wherein the controller is further configured to provide an indication of a non-disinfected state via the notification system when the enclosure is adjusted from the closed position to the open position during the predetermined second duration.

5. The device of claim 1, wherein the enclosure includes a locking mechanism configured to prevent adjustment of the enclosure from the closed position to the open position and the controller is further configured to lock the enclosure in the closed position via the locking mechanism when the ultraviolet light source is illuminated.

6. The device of claim 5, wherein the enclosure includes a locking override mechanism and the controller is further configured to:
in response to an actuation of the locking override mechanism while the enclosure is in the closed position and the ultraviolet light source is illuminated, discontinue illumination of the ultraviolet light source and unlock the locking mechanism to permit adjustment of the enclosure from the closed position to the open position.

7. The device of claim 1, further comprising a notification system in communication with the controller, wherein the controller is further configured to provide an indication of a disinfected state via the notification system when the enclosure is maintained in the closed position following illumination of the ultraviolet light source for the first duration.

8. The device of claim 7, wherein the controller is further configured to provide an indication via the notification system when the ultraviolet light is illuminated.

9. The device of claim 1, wherein the ultraviolet light source comprises one or more ultraviolet light-emitting diodes; wherein the enclosure is composed of an ultraviolet light shielding material; wherein the medical device comprises a catheter; and wherein the predetermined first duration is longer than the predetermined second duration.

10-12. (canceled)

13. The device of claim 1, further comprising a data-holding system in communication with the controller, wherein the controller is further configured to:
for each adjustment of the enclosure from the closed position to the open position, associate an access timestamp with said adjustment and store the access timestamp in the data-holding system and, if said adjustment is performed during the predetermined first duration, associate a flag with the access timestamp and store the flag in the data-holding subsystem.

14. The device of claim 13, wherein the controller is further configured to send the access timestamps and flags stored in the data-holding system to a remote computing device.

15. A device for maintaining disinfection of access sites of medical devices, comprising:
a receiving module comprising an enclosure configured to engage an end of a medical catheter line such that an access site of the line is positioned within a chamber of the enclosure, the enclosure adjustable between at least a closed position and an open position, wherein in the closed position the enclosure encloses the access site within the chamber and wherein in the open position the enclosure exposes the access site, wherein a light source is positioned to direct light either directly or indirectly to the access site when in the closed position.

16. The device of claim 15, wherein a twist-cap is provided to form the closed position.

17. The device of claim 16, wherein a squeezable cap is provided to form the closed position.

18. The device of claim 15, wherein a cap is provided to form the closed position, the cap including a recess to hold a line external to the enclosure.

19. The device of claim 15, further comprising a releasable adhesive colloid hydrogel patch for attachment to a patient's skin.

20. The system formed by multiple of the device of claim 17, wherein each of the multiple devices is releasably coupled to each other to form an array of varying size.

21. The device of claim 15, further comprising a controller,
wherein the controller comprises:
a logic subsystem; and
a data-holding subsystem comprising machine-readable instructions stored thereon that are executable by the logic subsystem to:
in response to an adjustment of the enclosure from the open position to the closed position:
illuminate the ultraviolet light source for a predetermined first duration while the enclosure is maintained in the closed position.

22. The device of claim 21, wherein the instructions further include instructions to:
following illumination of the ultraviolet light source for the first duration, illuminate the ultraviolet light source for a predetermined second duration at predetermined time intervals while the enclosure is maintained in the closed position; and
in response to an adjustment of the enclosure from the closed position to the open position:
discontinue illumination of the ultraviolet light source; and
provide an indication of a non-disinfected state if said adjustment occurs during the first duration.

23. The device of claim 22, wherein the data-holding subsystem comprising machine-readable instructions stored thereon are further executable by the logic subsystem to detect the receiving module and update the predetermined first duration and the predetermined second duration based on the detection of the receiving module.
